(19) United States
(12) Patent Application Publication
(10) Pub. No.: US 2010/0069607 A1
(43) Pub. Date: Mar. 18, 2010

(54) METHOD OF COVALENTLY LINKING A CARBOHYDRATE OR POLYALKYLENE OXIDE TO A PEPTIDE, PRECURSORS FOR USE IN THE METHOD AND RESULTANT PRODUCTS

(75) Inventors: Derek MacMillan, London (GB); Javier Blanc, Barcelona (ES)

Correspondence Address: SENNIGER POWERS LLP, 100 NORTH BROADWAY, 17TH FLOOR, ST LOUIS, MO, 63102, US

(73) Assignee: UCL BUSINESS PLC, London (GB)

(21) Appl. No.: 12/306,885

(22) PCT Filed: Jun. 29, 2007
(86) PCT No.: PCT/GB2007/002438

§ 371 Date: Dec. 1, 2009

(30) Foreign Application Priority Data
Jun. 30, 2006 (GB) 0613147.8

(51) Int. Cl. C07K 17/10; C07K 14/00; C08B 37/00; C08H 1/00

(52) U.S. Cl. 530/322; 530/395; 536/53; 530/409; 530/324

(57) ABSTRACT

A glycopeptide of the formula S-L-X—P, wherein: S is selected from an optionally protected monosaccharide, an optionally protected polysaccharide, a polyalkylene oxide chain and a group of the formula II, wherein R₁ and R₃ are independently selected from H or Ac, R₄ is Ac, and R₂ is a group of formula IV, wherein R₇ and R₈ are each independently selected from an optionally protected monosaccharide and an optionally protected polysaccharide, A, B, C and D are each independently 1 or 2, and m is 1 to 5; -L- is a moiety of the formula III wherein R₅ and R₆ are independently selected from H and Me and n is 1 to 3; and P is a peptide chain containing at least one amino acid having on its side chain the atom X, wherein X is an oxygen, a sulphur atom or a —CH₂— moiety.

The present invention relates to a method of covalently linking a carbohydrate or polyalkylene oxide to a peptide to form a glycopeptide-like compound, precursor compounds for use in the method and the resultant products.

Glycopeptides occur widely in nature. Many hormones are glycoproteins and they can also be found in cell walls. Naturally occurring glycopeptides have one or more carbohydrate groups directly attached to a peptide via the side chain of an amino acid. The bond that links the initial saccharide of the carbohydrate to the amino acid side chain is a glycosidic bond. Two general types of glycosidic bond occur naturally in glycopeptides: the O-glycosidic bond and the N-glycosidic bond, depending on whether the atom of the amino acid attached to the initial saccharide of the carbohydrate is O (such as in serine or threonine) or N (such as in asparagine). The term for the attachment of carbohydrates to proteins in cells is glycosylation.

The biological role of the carbohydrates in glycoproteins is not yet fully understood. It is known that the attachment of oligosaccarides to a protein chain alters its hydrophobicity. Additionally, it has been suggested that steric interactions between the peptide and oligosaccharide may favour one folding route over another. It has also been found that deglycosylated proteins (i.e. naturally occurring glycopeptides having had the saccharide moieties removed) generally do not survive in the body for long compared to the glycosylated analogues.

Some naturally occurring glycoproteins are only produced in very small quantities in animals. An example of such a protein is erythropoietin (EPO), which is produced in kidneys of mammals. EPO is required for the production of red blood cells in a process called erythropoesis. It acts by stimulating precursor cells in the bone marrow causing them to divide and differentiate into red blood cells. Only minute quantities of naturally-occurring EPO can be derived from the urine of a mammal. Because of the low availability of natural EPO, researchers developed other methods of its production, one of the most commercially successful of which has been producing EPO using DNA recombinant techniques to express the protein in a host organism, such as engineered tissue culture systems.

Recent studies have been directed to chemically synthesising glycoproteins and glycoprotein-like structures, as opposed to using recombinant DNA methods, to reproduce or mimic naturally occurring glycoproteins or to explore in greater depth the role of the carbohydrates in glycoproteins. It has been found that there are significant difficulties in the chemical synthesis of glycoproteins having O- and N-glycosidic bonds since the carbon in the saccharide attached to the ‘O’ or ‘N’ of the amino acid is chiral in naturally-occurring glycopeptides, so any method of linking the particular amino acid with the carbohydrate should, ideally, be stereoselective and reproduce the same chirality at this carbon centre. Further difficulties are generally found in the extensive protection and deprotection required for oligosaccharides.

It has been found that some polymeric chains may be attached to a peptide backbone in place of or in addition to carbohydrates and that the resultant glycopeptide-like structures successfully mimic the naturally-occurring glycosylated analogues. One such polymeric chain is polyethylene glycol (PEG). Examples of glycoprotein-mimics having polyethylene glycol chains attached may be found in International Patent Publication WO 01/02017 and EP 1 064 951 A2. These publications both disclose the ‘PEGylation’, i.e. the attachment of PEG, to an EPO peptide (in addition to the existing carbohydrate groups) using various types of linker.

A total chemical synthesis of a PEGylated EPO-mimic is disclosed in Chemistry and Biology, Vol. 12, 371-383, March 2005 and Science, Vol. 299, 884-887, March 2006. Various PEG chains were attached to a peptide backbone similar to that occurring in nature via an oxime bond.

Deiters et al disclose in Bioorg. Med. Chem. Lett. 14 (2004) 5743-5745 a method of expressing a protein containing an unnatural amino acid, para-azidophenylalanine, in yeast and then attaching the azide group of this amino acid to an alkyne-bearing PEG group. This method, while allowing for the site-specific attachment of a PEG group to a protein, has the disadvantage that it requires the expression of the protein in an organism. It is therefore very difficult to apply the technique to other proteins of interest.

While PEGylated glycopeptide mimics have had some success, there has been a limited amount of development of glycopeptides having carbohydrates attached. This is due in part to the ease of working with PEG, since it does not require the protection and deprotection of carbohydrates, nor does it have a chiral centre to further complicate the synthesis. There is thus a desire to develop methods of attaching carbohydrates to peptides, to allow for the research into the chemistry and biology of glycopeptides.

The most common method of chemically synthesising peptides in high yields is solid phase synthesis. In solid phase synthesis a peptide chain is constructed by adding one amino acid at a time to the growing peptide chain. The C-terminus of the peptide chain is removably attached to the solid support and each new peptide is added at the N-terminus of peptide chain. There are two types of solid phase synthesis methods: ‘FMOC’ synthesis and ‘tBOC’ synthesis, so called because of the ‘FMOC’ and ‘tBOC’ protecting groups, respectively, used on the N-terminus of the peptide. These protecting groups are removed before the attachment of each amino acid to, the existing peptide chain on the solid phase. Fmoc synthesis has been found to be generally more appropriate for peptides containing post translational modifications such as glycosylation and phosphorylation. Peptides assembled using BOC chemistry are normally cleaved with HF (hydrogen fluoride) which is not compatible with acid labile glycosidic linkages.

Solid phase synthesis can be used to construct peptides having up to about 50 amino acids efficiently, but beyond this, the yield of the peptides constructed are too small to be commercially viable. To create larger peptide chains, a technique called native chemical ligation has been developed; the technique is used to couple peptide chains together. It requires one peptide having a C-terminus thioester and the other peptide having a cysteine at its N-terminus. An example reaction illustrating the coupling is shown below.

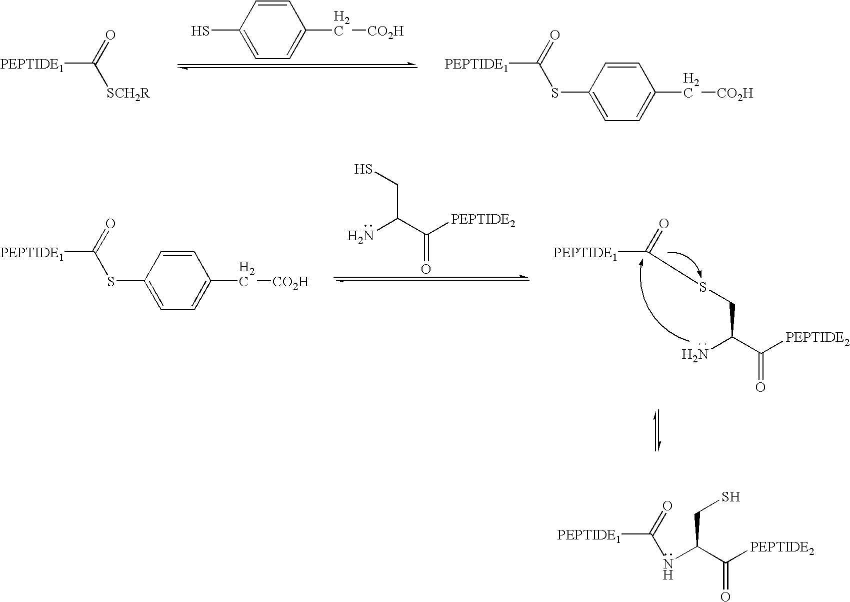

In creating glycopeptides using synthetic chemical techniques, it is therefore necessary for any technique to be compatible with solid phase synthesis and native chemical ligation. In other words, if a saccharide moiety is chemically bonded to a peptide chain using a particular linkage during solid phase synthesis, the linkage used should not break either when further solid phase synthesis is carried out or the peptide is subjected to native chemical ligation, including the typical protection and deprotection conditions required. The linkages formed must be able to withstand both acidic and basic conditions to which they may be exposed in solid phase synthesis, particularly FMOC solid phase synthesis. Additionally, they must be able to withstand the conditions for forming the peptide-peptide bonds in native chemical ligation. For example, while some carbohydrate-peptide linkages in the prior art include disulphide bonds it has been found that these are susceptible to degradation under native chemical ligation conditions.

The present inventors have previously disclosed a method of attachment of a saccharide to the cysteine residue of a synthetic peptide chain using glycosyl iodoacetamides, which is suitable for use in Fmoc solid phase synthesis of peptide and native chemical ligation (Macmillan et al, Org. Lett. 2002, 4, (9), 1467-1470). The method is particularly suitable for synthesising peptides having a number of cysteine residues, each of which may a different role in the ultimate peptide product, e.g. one cysteine may be for attachment of the saccharides and another cysteine may be for use in the formation of disulphide bonds. The peptide chain synthesised in this paper included two cysteine groups, only one of which was to be attached to a saccharide. The sulphur atom of each cysteine group was attached to an orthogonal protecting group (a different protecting group was attached to each sulphur atom, so that each could be removed independently of the other.) Glycosyl iodoacetamides are generally difficult to prepare, since in their preparation the process of their synthesis requires hydrogenation and then acylation. They are generally unstable to further manipulations, e.g. addition of other saccharides, as they are readily susceptible to attack by nucleophiles and are unstable to light.

The present invention aims to overcome or mitigate at least some of the problems associated with the techniques of the prior art.

Accordingly, in a first aspect, the present invention provides a compound for linkage to a peptide, the compound having the formula I

S-L-Hal formula I

wherein S— is a moiety of the formula II or a polyalkylene oxide chain

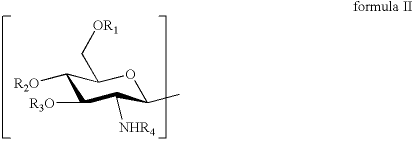

-L- is a moiety of the formula III

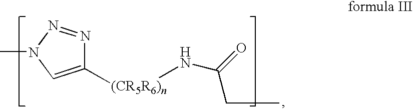

### and Hal is Br or I;

wherein R₁ and R₃ are independently selected from H and Ac,

### R₄ is Ac,

R₅ and R₆ are independently selected from H and Me,
n is 1 to 3, and
R₂ is a group selected from H, an optionally protected monosaccharide, an optionally protected polysaccharide, Ac, and a group of the formula IV,

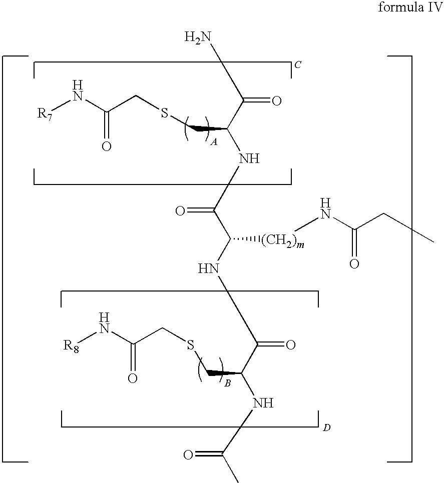

wherein R₇ and R₈ are each independently selected from an optionally protected monosaccharide and an optionally protected polysaccharide,
A, B, C and D are each independently 1 or 2,
m is 1 to 5.

In a second aspect, the present invention provides a method of synthesising a compound of the first aspect of the invention, the method comprising:

contacting, in the presence of a suitable catalyst, a compound of the formula V, wherein S is as defined in the first aspect,

S—N₃ formula V

with a compound of the formula VI

HC≡C-L^{p}-Hal formula VI,

wherein -L^{p}- is a moiety of the formula VII

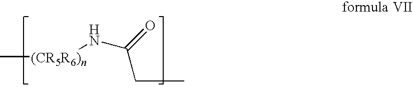

wherein R₅, R₆, n and Hal are as defined in the first aspect of the invention.

In a third aspect, the present invention provides use of the compound of formula I, S-L-Hal, in the synthesis of a glycopeptide, wherein S is selected from an optionally protected monosaccharide, an optionally protected polysaccharide, a group of the formula II wherein R₂ is a group of formula IV as defined above, and a polyalkylene oxide chain.

In a fourth aspect, the present invention provides a glycopeptide of the formula S-L-X—P, wherein S is selected from an optionally protected monosaccharide, an optionally protected polysaccharide, a group of the formula II, wherein R₂ is a group of formula IV as defined above, and a polyalkylene oxide chain, L is a moiety as defined in the first aspect of the invention and P is a peptide chain containing at least one amino acid having on its side chain the atom X, wherein X is an oxygen or a sulphur atom, or a —CH₂— moiety.

In a fifth aspect, the present invention provides a method of synthesising a glycopeptide as defined in the fourth aspect of the invention, the method comprising:

contacting a peptide of formula H—X—P with a compound of formula S-L-Hal, in the presence of a base to form S-L-X—P, wherein X, P, S and L are as defined herein.

In a sixth aspect, the present invention provides a method of synthesising a glycopeptide as defined in the fourth aspect of the invention, the method comprising contacting a compound of the formula S—N₃ with a compound of the formula HC≡C-L^{p}-X—P in the presence of a suitable catalyst, wherein S, L, X and P are as defined herein.

In a seventh aspect, the present invention provides a method of synthesising a compound of the formula HC≡C-L^{p}-X—P, wherein -L^{P}- and P are defined as in the second aspect, and wherein X is an oxygen or sulphur atom, the method comprising:

contacting an amino acid having at least one atom X on its side chain, with a compound of formula HC≡C-L^{p}-Hal, wherein Hal is Br or I, to form a HC≡C-L^{p}-X-functionalised amino acid, and using said functionalised amino acid in peptide chain assembly to form HC≡C-L^{p}-X—P.

In an eighth aspect, the present invention provides a method of synthesising a compound of the formula HC≡C-L^{p}-X—P, wherein -L^{P}- and P are defined as in the second aspect and wherein X is an oxygen or sulphur atom, the method comprising:

providing a peptide chain P, wherein P is a peptide chain containing at least one amino acid having on its side chain the atom X, wherein X is an oxygen or sulphur atom,

and contacting the peptide chain with a compound of formula HC≡C-L^{p}-Hal, wherein Hal is Br or I, to form HC≡C-L^{p}-X—P.

Preferred features of the invention may be found in the description below and in the dependent claims.

“Glycopeptide” in the above aspects and from hereon means a peptide having attached thereto one or more saccharides or a polyalkylene oxide group via a linker moiety.

The present inventors have surprisingly found that the moiety -L- as defined above provides a very stable linkage between a saccharide/polyalkylene oxide chain and the oxygen or sulphur atom on the side chain of an amino acid of a peptide (e.g. the sulphur atom of cysteine), so stable in fact that it can withstand all the conditions to which one would normally subject a peptide during solid phase synthesis and native chemical ligation, including the addition and removal of protecting groups. The precursors that may be used in the present invention, for example the azides (e.g. S—N₃, as defined herein), have been found to be much simpler to prepare, are stable to light and, perhaps most importantly, can be adapted as necessary with many synthetic transformations. The azide precursors can be reacted with acetylenes to form triazole groups in high yields under mild conditions (e.g. under aqueous conditions at 37° C. in the presence if Cu(I) ions), while not affecting any other typical protecting groups that may be present on the moiety attached to the azide (e.g. carbohydrate groups) or on any peptides (if present in the reaction). Further, the compounds of the present invention may be attached to suitable amino acids (particularly cysteine) of synthetic or recombinant proteins of potentially any size. Additionally, large ‘S’ groups can be attached to the peptide. The method of the present invention has been found to be a much more efficient synthetic route to peptides having oligosaccharides or polyalkylene oxide chains attached compared to methods of chemically synthesising naturally-occurring glycopeptides, i.e. those having O-glycosidic bonds and/or the N-glycosidic bonds as described above.

To the inventors' knowledge, the linker L has not been used to join a peptide with a saccharide or polyalkylene oxide chain. The method of the present invention also has the advantage that it does not require expression of the peptide in an organism (as in Deiters et al above) and therefore allows greater flexibility in the construction of the peptides, while still being able to direct with reasonable certainty where the saccharides or polyalkylene oxide chains are attached on the peptide chain.

Native chemical ligation has been shown to fail when trying to join a glycopeptide having large oligosaccharide moieties near the ligation site with another peptide. It is believed that the method as disclosed in the sixth aspect of the present invention may overcome such difficulties since native chemical ligation can be carried out on proteins having acetylenes attached (i.e. of the formula HC≡C-L^{p}-P) and the azide attachment of the saccharides (i.e. the reaction with the compounds of the formula S—N₃) can be carried out in solution following native chemical ligation.

The present invention will now be further described. In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

In formula II above, R₂ may be H or Ac. Alternatively, R₂ may be an optionally protected monosaccharide selected from glucose, glucosamine, galactose, N-acetylglucosamine, galactosamine, mannose, fucose and sialic acid. Of those, optionally protected galactose and glucoseamine are preferred.

R₂ may be an optionally protected polysaccharide containing from 2 to 5 component sugars and comprising one or more of glucose, glucosamine, galactose, N-acetylglucosamine, galactosamine, mannose, fucose and sialic acid.

Preferably, the component sugar(s) of the mono or polysaccharides are D-sugars. Preferably all component sugars are β-anomers.

“Protected monosaccharide” and “protected polysaccharide” means that each oxygen (which in its free state would be a hydroxyl group) of the component sugars is attached to a protecting group. The protecting group is preferably an acetyl group, Ac.

If R₂ is a saccharide, preferably the bond between this saccharide and the N-acetylglucosamine structure in formula I is a 1,4′ linkage.

In each polysaccharide, preferably the linkage between each component sugar is a 1,4′ linkage.

R₂ may be a moiety of formula IV. Preferably, A and/or B is 1. If C or D is 2, then formula IV may comprise two groups of R₇ or R₈ respectively. If formula IV comprises two R₇ groups, these two R₇ groups may be different, but are preferably are the same. Likewise, if formula IV comprises two R₈ groups, these two R₈ groups may be different, but are preferably are the same. R₇ may be the same as or different from R₈.

R₂ may be a moiety of formula IVA.

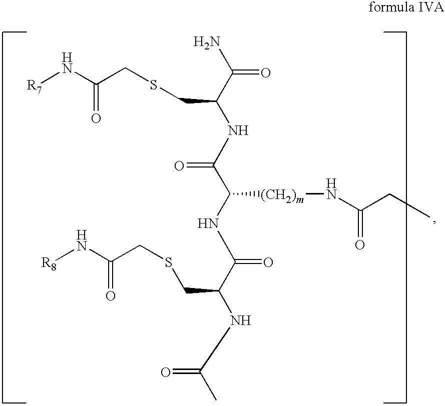

wherein R₇ and R₈ are each independently selected from an optionally protected monosaccharide and an optionally protected polysaccharide and m is 1 to 5.

R₇ and/or R₈ may be an optionally protected polysaccharide containing from 2 to 5 component sugars and comprising one or more of glucose, glucosamine, galactose, N-acetylglucosamine, galactosamine, mannose, fucose and sialic acid.

R₇ and/or R₈ may be an optionally protected disaccharide comprising one or more of glucose, glucosamine, galactose, galactosamine, mannose, fucose and sialic acid.

R₇ and/or R₈ may be a disaccharide comprising N-acetylglucosamine and galactose, wherein galactose is the terminal sugar component of the disaccharide.

In formula III, preferably n is 3.

“Polyalkylene oxide chain” preferably comprises a polyethylene oxide chain. The polyethylene oxide chain may contain 450 to 900 ethylene oxide units, preferably 500 to 800 ethylene oxide units, more preferably 650 to 700 ethylene oxide units.

In the methods of the second and sixth aspects, the catalyst preferably comprises Cu(I) or Cu(II), preferably Cu(I). The method may comprise providing a substance containing Cu(II) (e.g. copper sulphate) in the presence of a reducing agent to form Cu(I) in situ to catalyse the reaction between the azide and the acetylene groups. The reducing agent preferably comprises sodium ascorbate and optionally tris-carboxyethylphosphine (TCEP). Alternatively a compound containing Cu(I), such as CuI or CuBr, may be added to the reaction mixture.

Alkylene oxide azides for use in the present invention are known. They may be synthesised as demonstrated in Tetrahedron Lett. 44(6) 2003, 1133-1135 and Chemical Communications, 2006, 1652-1654.

In the third aspect of the present invention, S— may be as defined herein.

In the third aspect of the present invention, at least part of the peptide chain of the glycopeptide may be synthesised using solid phase synthesis and/or native chemical ligation. Preferably the part of the peptide to which one or more S-L- moieties will be attached will be synthesised using solid phase synthesis, preferably FMOC solid phase synthesis. The native chemical ligation may be carried out before or after attachment of the S— moiety to the peptide chain via the linker L.

The fourth aspect provides a glycopeptide of the formula S-L-X—P, as defined above. The at least one amino acid is preferably cysteine or homocysteine. The peptide chain may comprise two or more amino acids having the atom X on its side chain, each attached to a moiety of the formula S-L-. One, two, three or four S-L groups, for example, may be attached to the peptide P via the X groups of amino acids. The peptide may comprise a cysteine at its N-terminus and/or a thioester at its C-terminus for attachment to a second peptide and/or third peptide using native chemical ligation. The second and/or third peptide may be a synthetic peptide (e.g. having been made in solid phase synthesis) or a peptide which has been made using recombinant techniques in an organism.

In the glycopeptide of the present invention, S is preferably as defined in the first aspect of the present invention. It has been found that such glycopeptides may be able to mimic naturally occurring N-linked class of glycoproteins, i.e. proteins in which the saccharides are attached to a peptide via an N-glycosidic bond.

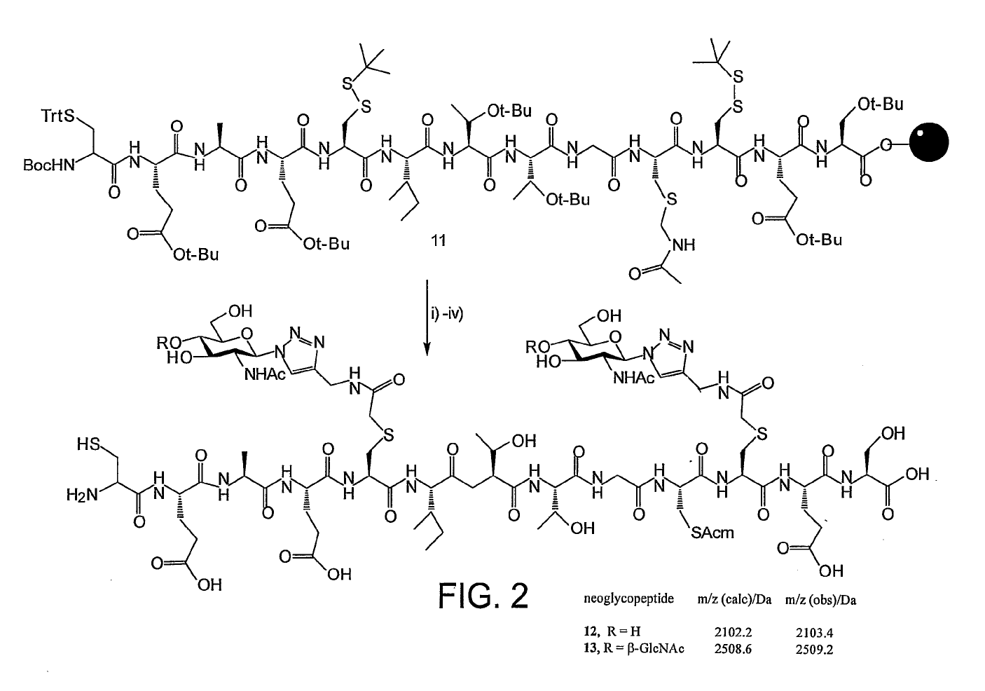

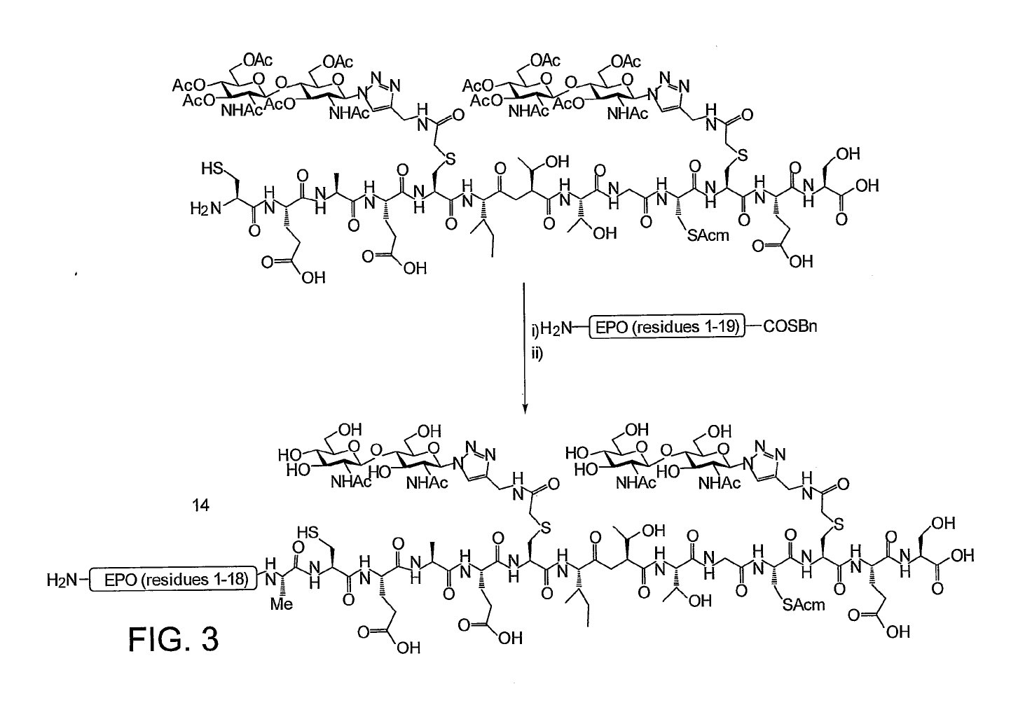

The glycopeptide may be a peptide as shown in FIG. 2 (either one of compounds 12 or 13) or peptide as shown in FIG. 3 (compound 14).

The base in the fifth aspect of the invention may comprise one or more of diisopropylethylamine, pyridine and triethylamine.

The methods of the fifth and sixth aspects may be carried out while the peptide P is attached to a solid support (for synthesising a peptide in solid phase synthesis) or while in solution. The species S may be attached to peptide of any length and of any sequence, as long as the relevant reactant groups in the peptide are available (e.g. the group HX—, such as HS— in cysteine) for reaction. Preferred solid supports include: Novasyn TGT, PEGA and fink amide resins. The methods of the fifth and sixth aspect may further comprise a first step of synthesising the peptide in solid phase synthesis, which may involve FMOC or tBOC protection of the N-terminus of the peptide during the synthesis.

The peptide synthesised in the solid phase may have substantially the same amino acid sequence as at least part of a naturally occurring glycopeptide, except that the amino acid(s) to which the saccharides groups would be attached in the naturally occurring glycopeptide are instead optionally protected cysteine or homocysteine (and one of the other amino acids in the natural sequence may be replaced with a cysteine that forms the N-terminus of the peptide chain, for native chemical ligation, if necessary). If the peptide synthesised in the solid phase represents only part of a naturally occurring glycopeptide (except for cysteine or homocysteine replacements), the remaining part(s) of the naturally occurring peptide sequence may be attached to it using techniques such as native chemical ligation, which may be before or after the S-L- moiety has been attached to the replacement cysteine(s) or homocysteine(s) of the peptide synthesised in the solid phase. This/these remaining part(s) of the peptide may be synthesised using solid phase synthesis and/or expressed in an organism using recombinant techniques known to the skilled person.

The peptide chain synthesised in the solid phase synthesis preferably comprises a first optionally protected cysteine residue at its N-terminus (for ligation of the peptide to a further peptide in native chemical ligation), and one or more further optionally protected cysteine or homocysteine residues (for attachment to one or more S-L- moieties). Preferably, all cysteine/homocysteine residues are protected during construction of the peptide during solid phase synthesis. Preferably, the protecting group on the first cysteine (at the N-terminus) is different from the one or more further cysteine or homocysteine residues in the peptide. The protecting group for the first cysteine may comprise the Trt group. The protecting group for the one or more cysteine or homocysteine residues may comprise an alkyl sulphide group, preferably, the —S-tBu group.

The constructed peptide comprising the protected N-terminus cysteine and protected one or more further cysteine or homocysteine residues may be subjected to a treatment that removes the protecting groups from the one or more further cysteine or homocysteine residues, but not the protecting group from the N-terminus cysteine. If the protecting group on the one or more other cysteine/homocysteine groups comprises the —S-tBu group, preferably this is removed by exposing the peptide to dithiothreitol and DIPEA, and/or one or more of ammonium carbonate, DTT, and propanedithiol.

The deprotected one or more further cysteine/homocysteine residues may then be reacted with either (i) one or more HC≡C-L^{p}-Hal compounds to form a peptide attached to one or more acetylene groups, each via L^{p} (the resultant peptide being denoted as HC≡C-L^{p}-P) or (ii) one or more S-L-Hal compounds to form a peptide having one or more S-L- moieties attached, each S-L moiety attached to a cysteine/homocysteine (the resultant peptide being denoted as S-L-X—P). The peptide HC≡C-L^{p}-X—P may be further reacted with the compound S—N₃ to form the peptide having one or more S-L- moieties attached, each S-L moiety attached to a cysteine/homocysteine residue (the resultant peptide being denoted as S-L-X—P). The resultant peptide may then be removed from the solid support using conventional techniques.

The protein formed in (i) or (ii) above, which has been removed from the solid support, may be combined with a second protein having a C-terminus thioester by native chemical ligation using conventional techniques. Such techniques are described in Science 2003, 299, (5608), 884-887 and below in the Examples. The native chemical ligation may be carried out in the presence of guanidine hydrochloride, mercaptoethanesulfonic acid (MESNA) and triscarboxyethylphosphine (TCEP). The second protein may also be a synthetic protein or a protein made using recombinant techniques.

The peptide HC≡C-L^{p}-X—P may be synthesised by direct incorporation of a suitably protected and functionalised amino acid comprising the HC≡C-L^{p} moiety in the peptide synthesis, for example in Fmoc solid phase synthesis. For example, an amino acid group having on its side chain the atom X, wherein X is an oxygen or sulphur atom may be reacted with HC≡C-L^{p}-Hal to form an HC≡C-L^{p}-X-functionalised amino acid. The functionalised amino acid may then be used in peptide chain assembly to form HC≡C-L^{p}-X—P.

When the peptide HC≡C-L^{p}-X—P is synthesised by direct incorporation of a suitably protected and functionalised amino acid comprising the HC≡C-L^{p}— moiety, the HC≡C-L^{p}- moiety may connected to the amino acid side chain by a —CH₂— moiety, i.e. X may be —CH₂—.

An example synthesis of peptide sequence similar to erythropoietin having PEG or saccharide attachments via the novel linkage of the present invention may be as follows. Further detail of various parts of the synthesis may be found in the Examples.

### Semi-Synthesis of Modified Erythropoietin (EPO)

### Target Sequence:

The amino acid alterations, relative to the Wild-type human EPO sequence, are underlined and the sulphur atom of the “C” residues are attached to attached PEG or saccharide chains, via the moiety -L-.

(SEQ ID No. 1)

(C)APP RLICDSRVLE RYLLEAKEAE CITTGCCESC SLNENITVPD
TKVNFYAWKR LEVGQQAVEV WQGLALLSEA VLRGQALLVK
SSQPWEPLQL HVDKAVSGLR SLTTLLRALG AQKEAISPPD
AAKAAPLRTI TADTFRKLFR VYSNFLRGKL KLYTGEACRT
GDR

(C) is an optional residue at the N-terminus.

### General Peptide Thioester Synthesis (Residues 1-32)

Peptide thioester synthesis was carried out using Rink linker modified amino PEGA resin. The peptide thioesters were prepared using the strategy described by the Unverzagt group.¹ Briefly, rink modified-PEGA resin (0.1 mmol) was deprotected by exposure to 20% piperidine in DMF. Fmoc-Phe-OH (5 equiv) was coupled using HBTU/HOBt as coupling reagents. The coupling time was 4 h. After Fmoc removal with 20% piperidine in DMF the sulfonamide linker was coupled through exposure of the resin to 3-carboxypropanesulfonic acid (50 mg, 0.3 mmol), HOBt (40 mg, 0.3 mmol) and DIC (47 μL, 0.3 mmol) for 5 h. The first amino acid (Fmoc-Ser(tBu)—OH, 5 equivalents per coupling) was then double coupled employing N-methylimidazole (40 μL, 0.5 mmol), DIC (78 μL, 0.5 mmol) as coupling reagents in 4:1 DCM/DMF for 16 h. The peptide was extended (target sequence: APP RLICDSRVLE RYLLEAKEAE CITTGCCES-SBn (SEQ ID No. 2)) in automated fashion using an Applied Biosystems model 433A peptide synthesiser and ultimately cleaved with benzylmercaptan, after ICH₂CN activation, using well established procedures.²

### General StBu Deprotection of the Assembled 32mer Peptide on Solid-Support.

DTT (100 mg) was dissolved in dry DMF (0.9 ml) and solid ammonium carbonate was added. After stirring for 5 minutes the supernatant was decanted and transferred to a peptide synthesis vessel containing resin-bound StBu protected peptide. (NOTE. The ammonium carbonate could be replaced by 2.5% v/v diisopropylethylamine). After 16 h the resin was filtered and washed exhaustively with DMF then DCM. The deprotection can be repeated if necessary.

### General Bromo/Iodoacetamide Couplings.

Iodo/bromoacetamides (of the formula S-L-Hal) (3. equivalents) were dissolved in 2.5% v/v pyridine in DMF (2.0 ml) and transferred to a peptide synthesis vessel containing resin-bound StBu deprotected peptide (50-100 mg). The reaction was allowed to proceed from 12-24 h in the absence of light. After this time, the resin was filtered and washed exhaustively with DMF then DCM.

It will be understood that the general StBu deprotection and General bromo/iodoacetamide couplings steps described above may be omitted if the synthetic peptides HC≡C-L^{p}-X—P have been synthesised by directly incorporating HC≡C-L^{p}-X— functionalised amino acids into the peptide chain synthesis.

### General TFA Cleavage and Purification.

Synthesis of the neoglycopeptide mimic prepared on Rink modified amino PEGA resin was routinely monitored, and ultimately deprotected upon exposure to 95% TFA, 2.5% ethanedithiol, 2.5% water for 3 h. After this time, the resin was filtered off and the filtrate was poured into ether (10 volumes). The precipitated peptide was then collected by centrifugation (10000 rpm, 5 mins (analytical), 3000 rpm, 15 min (preparative)). The precipitate was re-suspended in ether (5 volumes) and collected by centrifugation once again. The crude glycopeptide mimics were dissolved in 30% MeCN/water and loaded directly onto a semi preparative HPLC column (250 mm×10 mm) using a gradient of 5% to 95% acetonitrile (containing 0.1% TFA) over 50 minutes. Fractions containing the glycopeptide products were identified by mass spectrometry and lyophilised to obtain the purified products as fluffy white solids.

Method for Production of Residues 33-166 (from E. coli)

His₁₀-fusion proteins were overexpressed from the commercially available (Novagen) pET16-b expression vector and purified by Nickel affinity chromatography according to the manufacturers instructions. Samples of each fraction obtained from Ni²⁺ column were analyzed by SDS-polyacrylamide gel electrophoresis. Fractions were combined and dialyzed overnight at 4° C. against 4 liters of water in 8-10 kDa cut-off dialysis bags. Protein formed a white precipitate that was transferred to a 15.0 mL Falcon tube and pelleted by centrifugation at 4000 rpm for 15 minutes at 4° C. The white precipitates obtained were redissolved in 80% formic acid to a concentration of approximately 0.6 mgmL⁻¹. This was then transferred to a 10.0 ml round bottomed flask and 5 mg CNBr added. The reaction was then stirred under argon with the exclusion of light overnight. The formic acid was then removed under reduced pressure and the dry pellet was resuspended in a minimal volume (150 μL) of binding buffer (100 mM NaCl, 50 mM Tris.HCl; pH 8.0) containing 6 M guanidine HCl and reduced with 1 mM DDT for 45 minutes at 37° C. The efficiency of the CNBr cleavage was analysed by LCMS. Cleaved protein (1-3) was obtained as a white precipitate after overnight dialysis against 4 liters of water and centrifugation at 4000 rpm for 15 minutes. CNBr cleavage was also carried out using urea buffer instead of 80% formic acid. The white precipitates from dialysis after Ni²⁺ affinity chromatography were dissolved in 8M urea containing 0.3 M HCl and reaction with CNBr carried out as described above. After 22 hours the protein sample was reduced with 1 mM DDT (after pH adjustment to approx. 8 with conc. NaOH) and analyzed by LCMS. The cleaved protein precipitate was obtained after an overnight dialysis against water and centrifugation at 4000 rpm for 15 minutes.

### General Procedure for Ligation Reactions

Ligations were carried out using a modification of the procedure described by Kochendoerfer et al.³ The cleaved protein fragments (the pelleted material from the dialysis was used directly. HPLC purified samples were also tested with no obvious improvement in reaction yield or rate. Samples were dissolved in 300 mM Na phosphate buffer prepared in 6 M guanidine hydrochloride; pH 8.0. Tris-carboxyethylphosphine (TCEP) was added to a final concentration of 20 mM and 2-mercaptoethanesulfonic acid (MESNA) was added to a final concentration of 1% w/v. This solution was added to a lyophilised aliquot of the synthetic peptide thioester and the reaction mixture was agitated on a shaking platform under argon and monitored by LC-MS.

### Refolding of the Semi-Synthetic Erythropoietin

Protein samples were reduced, dialyzed against 6 M guanidinium hydrochloride, 50 mM Tris.HCl; pH 8.0 under a nitrogen atmosphere, diluted 1:50 (to approximately 1 M) and oxidatively refolded by dialysis against 2%; N-lauroylsarcosine, 50 mM Tris.HCl, pH 8.0, 40 μM CuSO₄.⁴ Refolded protein was then concentrated using a Centricon, centrifuge concentrator.

### REFERENCES FOR THE EXAMPLE SYNTHESIS

1) Mezzato, S.; Schaffrath, M.; Unverzagt, C., An orthogonal double-linker resin facilitates the efficient solid-phase synthesis of complex-type N-glycopeptide thioesters suitable for native chemical ligation. Angew. Chem. Int. Ed. 2005, 44, 1650-1654.
2) Shin, Y.; Winans, K. A.; Backes, B. J.; Kent, S. B. H.; Ellman, J. A.; Bertozzi, C. R., Fmoc-based synthesis of peptide-(alpha)thioesters: Application to the total chemical synthesis of a glycoprotein by native chemical ligation. J. Am. Chem. Soc. 1999, 121, (50), 11684-11689.
3) Kochendoerfer, G. G.; Chen, S.-Y.; Mao, F.; Cressman, S.; Traviglia, S.; Shao, H.; Hunter, C. L.; Low, D. W.; Cagle, E. N.; Carnevali, M.; Gueriguian, V.; Keogh, P. J.; Porter, H.; Stratton, S. M.; Wiedeke, M. C.; Wilken, J.; Tang, J.; Levy, J. J.; Miranda, L. P.; Crnogorac, M. M.; Kalbag, S.; Botti, P.; Schindler-Horvat, J.; Savatski, L.; Adamson, J. W.; Kung, A.; Kent, S. B. H.; Bradburne, J. A., Design and Chemical Synthesis of a Homogeneous Polymer-Modified Erythropoiesis Protein. Science 2003, 299, (5608), 884-887.
4) Boissel, J. P.; Lee, W. R.; Presnell, S. R.; Cohen, F. E.; Bunn, H. F., Erythropoietin structure-function relationships. Mutant proteins that test a model of tertiary structure. J. Biol. Chem. 1993, 268, (21), 15983-15993.

Embodiments of the present invention will now be further illustrated with reference to the Examples and the drawings, in which:

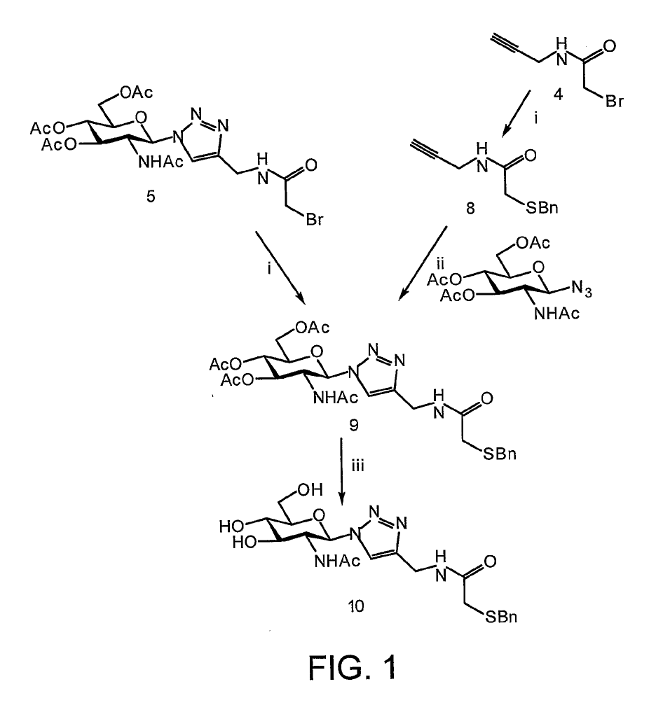

FIG. 1 illustrates various synthetic routes to a benzyl thioether (10). In the formation of 10, it is necessary to react the azide with an acetylene group to form the triazole and react the bromoacetamide group with the benzyl thiol to form the thioether linkage. The reaction demonstrates that either the benzyl triazole can be formed before the thioether linkage or vice versa. Reagents and conditions: i) BnSH, Et3N, DMF, 16 h, 84% and 75% for 8 and 9 respectively ii) sodium ascorbate (1.1 eq), Cu(II) SO4.5H2O (0.1 eq), CHCl3, EtOH, H2O (9:1:1), 37° C., 16 h, 91%, iii) 2% v/v hydrazine monohydrate EtOH, 72 h, 66%;

FIG. 2 illustrates the coupling of an example compound of the present invention (5 or 7) with a peptide chain to form an example peptide of the present invention. Reagents and conditions: i) 10% w/v DTT, 2.5% DIPEA, DMF, 16 h, ii) 5 or 7 (3 equivs per thiol), 2.5% v/v Et3N, DMF, 16 h, iii) 95% TFA, 2.5% ethanedithiol, 2.5% H2O, 4 h, iv) 2% v/v aqueous hydrazine monohydrate, 1 h;

FIG. 3 illustrates the native chemical ligation of the peptide produced in the scheme shown in FIG. 2 with a further peptide (EPO residues 1-19). Reagents and conditions i) 6 M guanidine HCl, 1% w/v MESNA, 300 mM Na phosphate buffer (pH 8.0), 10 mM TCEP, ii) 2% aqueous H2N—NH2;

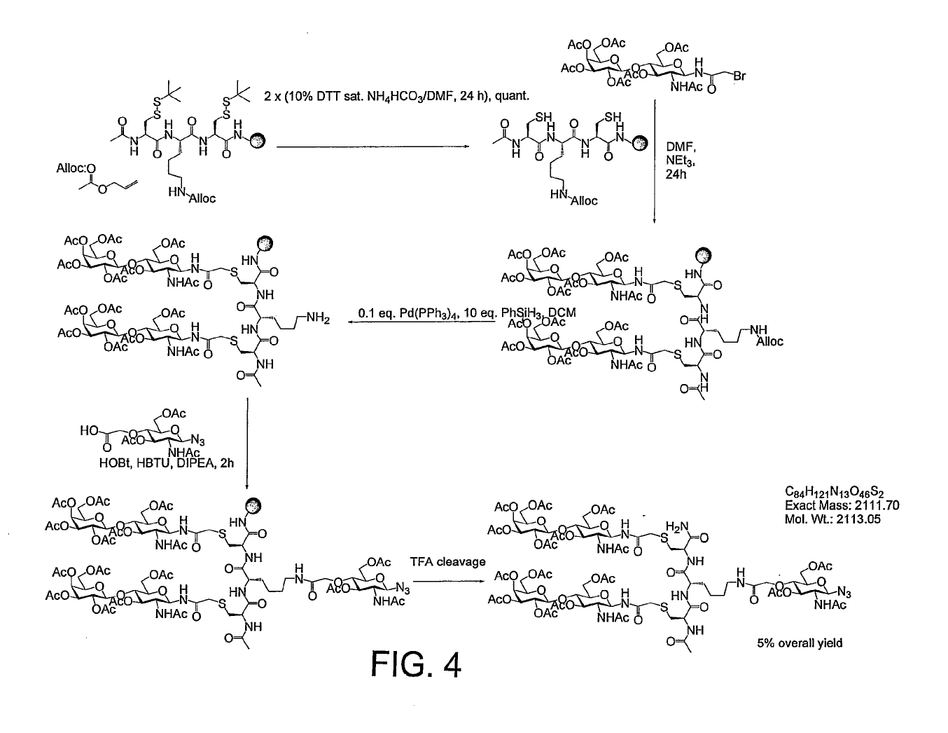

FIG. 4 illustrates the chemical synthesis of a double oligosaccharide compound (Compound A) terminating in an azide group for attachment to an acetylene;

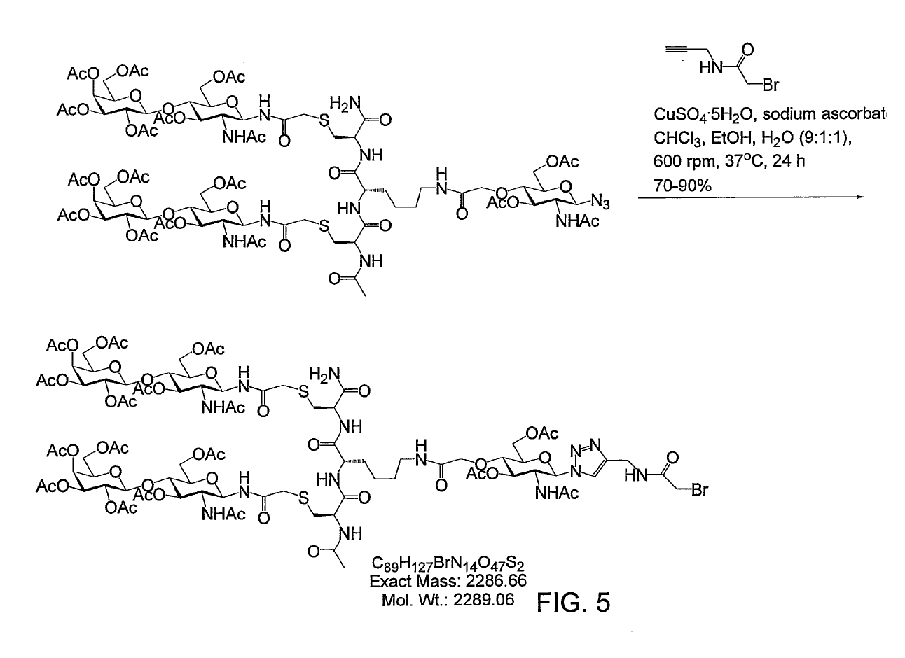

FIG. 5 illustrates the chemical synthesis of Compound B from Compound A, i.e. the triazole formation by the attachment of the propargyl bromoacetamide to Compound A;

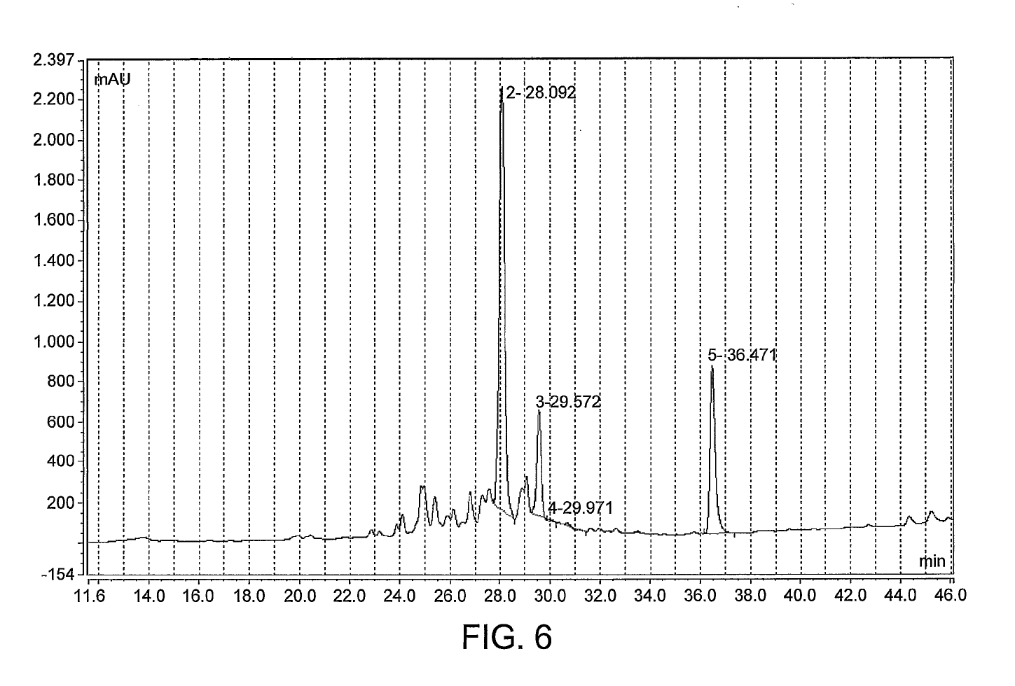

FIG. 6 shows HPLC of the crude peptide thioester (EPO (residues 1-19), for which ESI-MS of 29 min fraction (residues 1-19-SBn) Calcd mass=2320.7 Da , Observed mass=2321.6 Da;

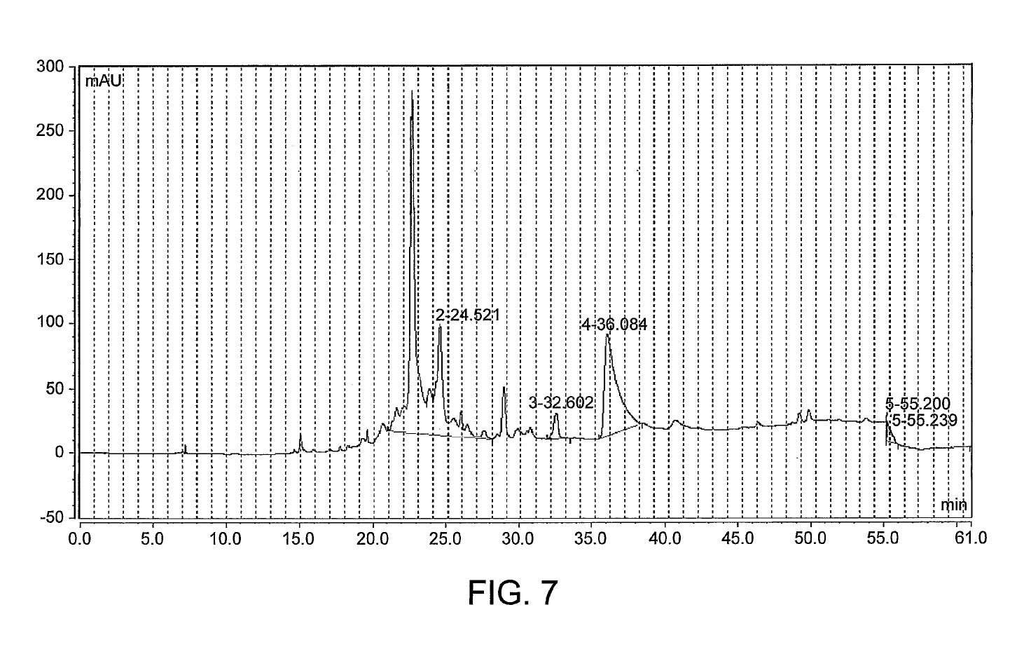

FIG. 7 shows the HPLC of crude compound 12 (prior to hydrazine deprotection). The peak in fraction 23 is the desired product;

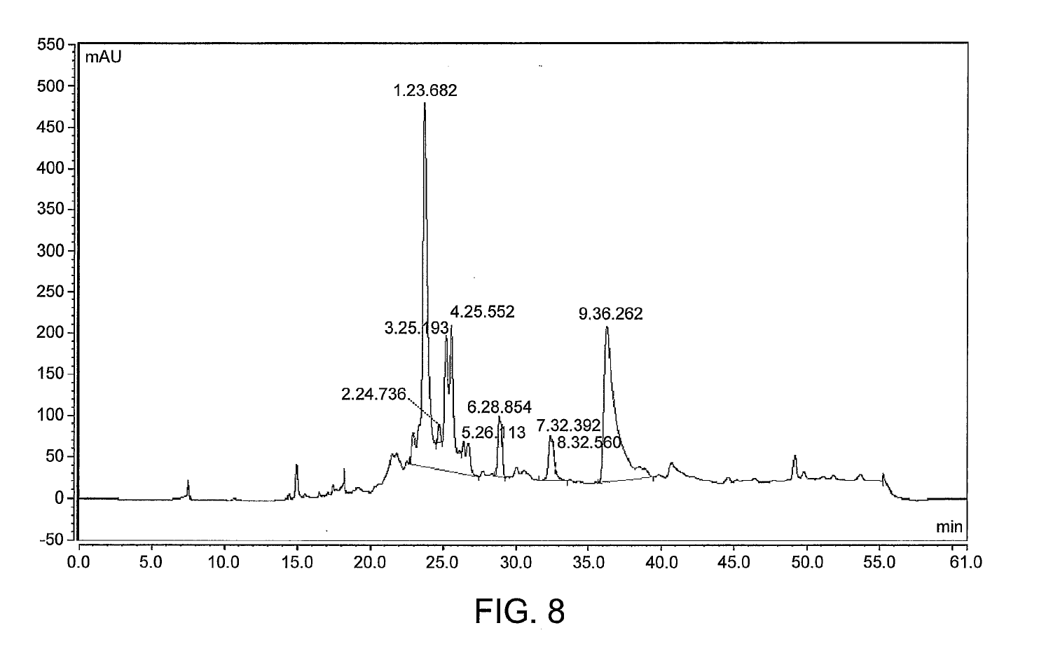

FIG. 8 shows the HPLC of crude compound 13 (prior to hydrazine deprotection). The major peak (retention time=23.7 min) is the desired product; and

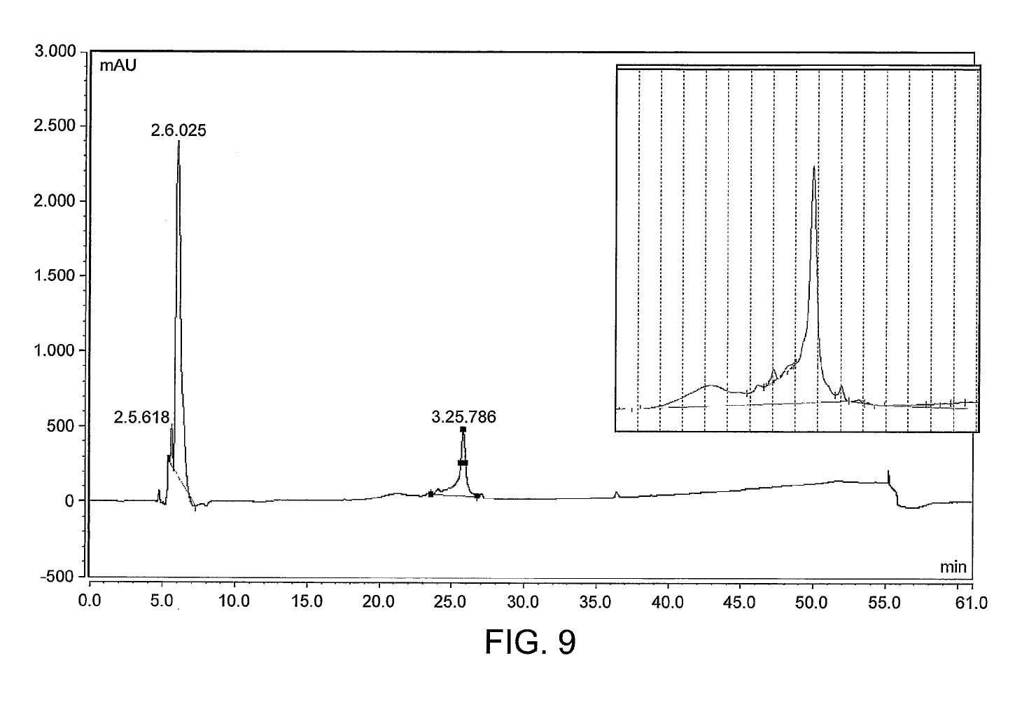

FIG. 9: The HPLC for compound 14 showed a single major species with a retention time of 25.8 mins which was confirmed as the desired product by ESI-MS (calculated Mwt=5125.6 Da, Obs MWt=5127.0 Da.

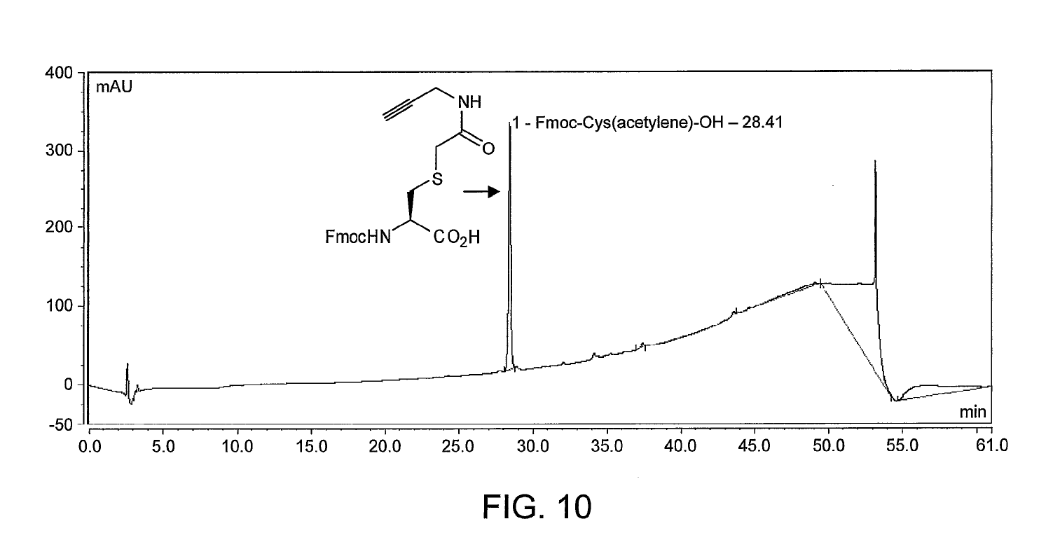

FIG. 10 shows the HPLC trace of purified N-α-(9-fluorenylmethoxycarbonyl)-L-Cysteine-S—(N-propargyl)carboxymethylamide

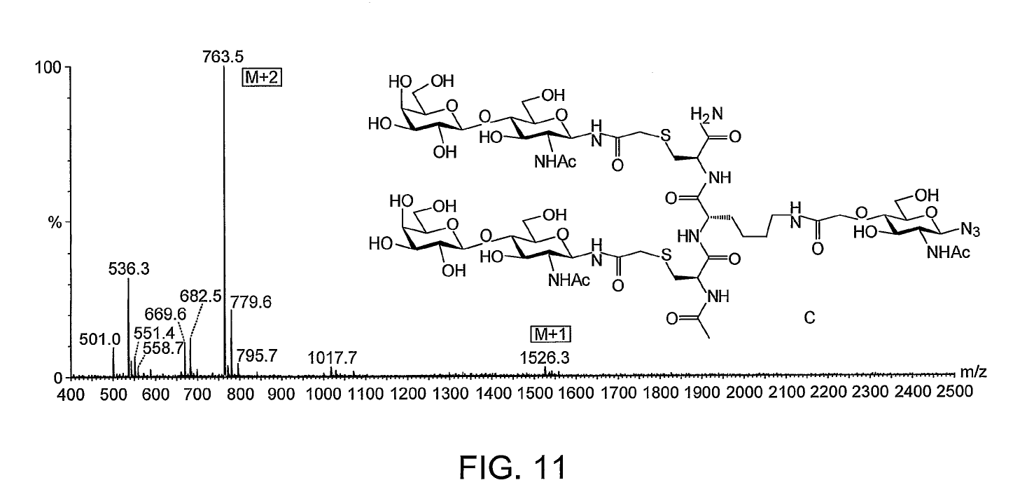

FIG. 11 shows the analytical data of compound C: C₅₆H₉₃N₁₃O₃₂S₂. MW: 1524.79. Found: (M+1) 1525.79; (M+2) 763.48; (M+2+NH₂NH₂) 779.40; 795.63 (M+2+2NH₂NH₂)

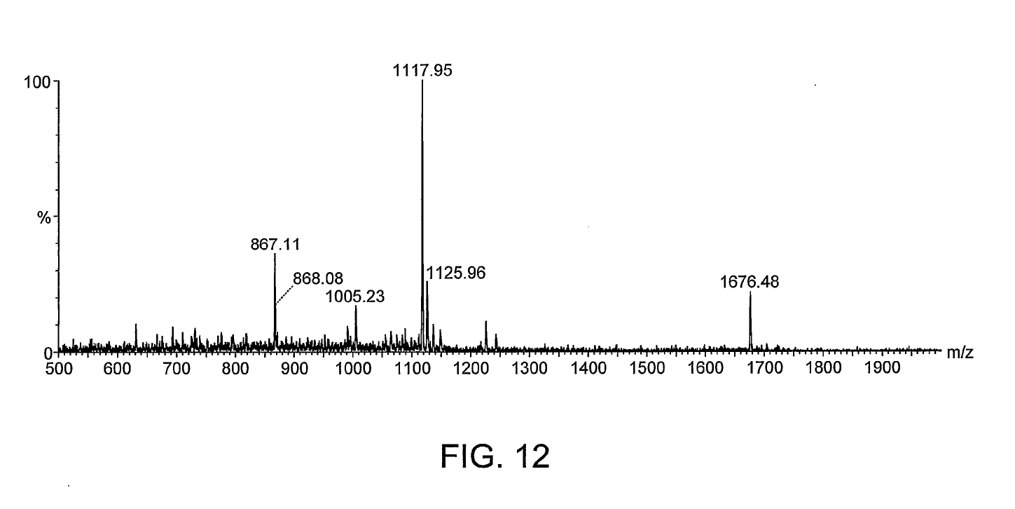

FIG. 12 shows the ESI-MS of HPLC of EPO 1-28-SBn thioester. Calculated m/z=observed m/z=2233.9

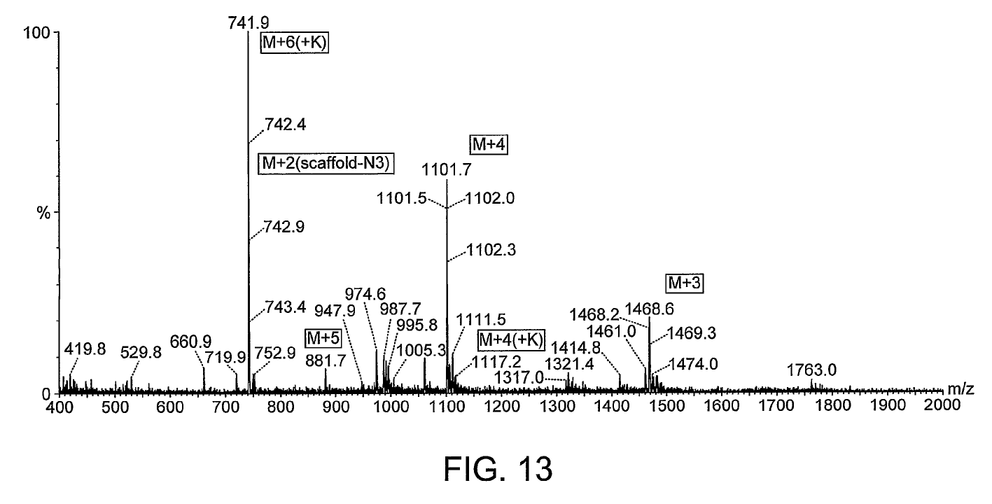

FIG. 13 shows the ESI-MS of: C₁₆₄H₂₆₈N₄₀O₈₄S₈. MW: 4400.6. Found: (M+3) 1468.6, (M+4 (+K)) 1111.5, (M+4) 1101.7, (M+5) 881.7, (M+2 (saccharide mimic-N₃)) 741.9.

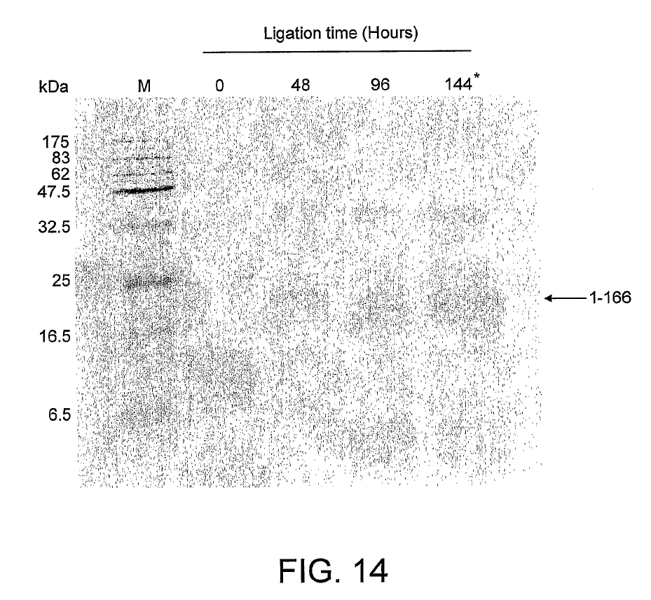

FIG. 14 shows protein ligation between EPO(1-28)SBn thioester (containing an acetylene at position 24) and bacterially derived EPO residued 29-166.

### EXAMPLES

### Example 1

In a first experiment, the present inventors prepared the peracetylaed glycopyranosylazides of N-acetylglucosamine (1) chitobiose (2) and N-acetyllactosamine (3), which are all constituents of the N-linked class of glycoproteins. The inventors then investigated conditions for their union with the heterobifunctional adaptor 2-bromoacetyl propargylamide (4) and the reaction of these saccharides with 4 proceeded smoothly under conditions reported in the recent literature, the 1,5-addition product being favoured by the presence of a Cu(I) catalyst (Table 1). The crude products did not require purification by column chromatography.

TABLE 1
Synthesis of neoglycopeptide precursor bromoacetamides

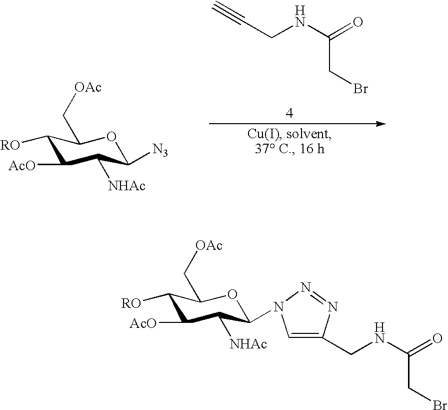

| | | | Yield |
|---|---|---|---|
| saccharide | catalyst | product | (%)^{c} |
| 1, R = Ac | Cu(I)I (5 eq)^{a} | 5 | 100 |
| 1, R = Ac | Cu(II)SO₄ (0.1 eq)^{b} | 5 | 97 |
| 2, R = (OAc)₄-β-Gal- | Cu(II)SO₄ (0.1 eq) | 6 | 91 |
| 3, R = (OAc)₃ β-GlcNAc- | Cu(II)SO₄ (0.1 eq) | 7 | 87 |

^{a}methanol as solvent;
^{b}active copper species generated in the presence of 1.1 equivalent sodium ascorbate in 9:1:1 CHCl₃/EtOH/H₂O as solvent;
^{c}isolated yield.

The inventors then aimed to expose the bromoacetamides to conditions typically encountered in peptide synthesis and native chemical ligation and, in particular, the conditions required to attach the linkages to the thiol groups of cysteine residues. Additionally, the inventors explored the possibility of reacting thiol groups directly with the bromoacetamide products 5-7 on solid phase (see FIG. 1) and the reaction of cysteine thiols with 4 such that ‘click’ chemistry, i.e. the attachment of the azides to the acetylenes, could subsequently be investigated in solution or on solid-phase with peptides displaying acetylenes.

4 and 5 reacted cleanly with benzyl mercaptan forming model thioethers 8 and 9 in 84% and 75% yield respectively. 8 also reacted cleanly with peracetylated 2-acetamido-2-deoxy-D-glucopyranosyl azide, affording 9 in 91% yield.

To establish whether the products might be stable to the usual acidic peptide cleavage conditions 9 was subjected to 95% aqueous TFA for 3 h. NMR analysis of the crude material after evaporation showed no decomposition had taken place.

Finally the acetyl esters were cleanly removed upon exposure to 2% v/v hydrazine hydrate in EtOH for 72 h and the fully deprotected compound 10 was obtained. Encouraged by the preliminary results the present inventors assembled a peptide fragment (11), similar in sequence to human erythropoietin (residues 21-32), plus an N-terminal cysteine residue, and furnished with two disulfide bond protected cysteine residues at pre-determined positions (see FIG. 2). The peptide was assembled using standard protocols for Fmoc solid-phase peptide synthesis and in automated fashion. The cysteine residues were deprotected on solid-phase by exposure to 10% w/v dithiothreitol (DTT) containing 2.5% v/v DIPEA to expose the thiol functional groups.

N-acetylglucosamine and the disaccharide chitobiose were then incorporated by exposure of the resin to bromoacetamides 5 or 7, employing three equivalents 5 or 7 per thiol in each reaction. After 16 h reaction at room temperature, cleavage of a small resin sample indicated that the reaction was complete as the starting material was not observed.

After cleavage from the solid support by treatment of the resin with 95% TFA, 2.5% ethanedithiol and 2.5% H2O for four hours the crude products were purified by semi-preparative HPLC, lyophilized, and treated with 2% v/v aqueous hydrazine hydrate containing 5% w/v DTT to obtain the fully deprotected products 12 and 13 in quantitative yield (determined by HPLC). Bromoacetamide 6 and acetylenic bromoacetamide 4 could also be incorporated into synthetic peptides in an identical fashion.

Fragment 13 was then coupled to a peptide thioester in a native chemical ligation reaction. The construction of the peptide thioester, corresponding to human erythropoietin residues 1-19, and its release from the solid support were monitored using the dual-linker approach recently described by Unverzagt and co-workers (Angew. Chem. Int. Ed. 2004, 44, 1650-1654).

In the ligation reaction equimolar quantities of each peptide were combined in 0.25 ml of 6 M guanidine hydrochloride containing 90 300 mM sodium phosphate buffer; pH 8.0, 1% w/v mercaptoethanesulfonic acid (MESNA) and 10 mM triscarboxyethylphosphine (TCEP) for 36 h with shaking at room temperature. After this time the reaction mixture was purified directly loading it onto a semi-preparative HPLC column. The ligated product (14) was the only species observed by HPLC.

In summary we have developed a novel class of neoglycopeptide that is compatible with modification of bacterially-derived cysteine mutant proteins, with synthetic peptides, and native chemical ligation. Furthermore the fusion of glycosyl azides with peptides.

### Experimental Details for Example 1

### Instrumentation

¹H NMR spectra were recorded at 250 and 300 MHz, ¹³C NMR spectra were recorded at 63 and 75 MHz and ¹⁹F NMR spectra were recorded at 235 MHz on a Bruker 250Y instrument. Chemical shifts (δ) were reported in ppm and coupling constants (J) in Hz, signals were sharp unless stated as broad (br), s: singlet, d: doublet, t: triplet, m: multiplet and q: quaternary. Residual protic solvent, CDCl₃ (δ_{H}: 7.26, s) was used as the internal standard in ¹H-NMR spectra unless otherwise stated. Electrospray mass spectroscopy was carried out on a Micromass Quattro LC electrospray with an applied voltage of 25-60V.

### Chromatography

Analytical TLC was carried out on Merck aluminium backed plates coated with silica gel 60F₂₅₄. Flash chromatography was carried out over Fisher silica gel 60 Å particle size 35-70 micron. Components were visualized using p-anisaldehyde dip and UV light (254 nm).

### Solvent and Reagents

All reagents and solvents were standard laboratory grade and used as supplied unless otherwise stated. Where a solvent was described as dry it was purchased as anhydrous grade. All organic extracts were dried over anhydrous magnesium sulphate prior to evaporation under reduced pressure.

### N-(propargyl)-bromacetamide (4)

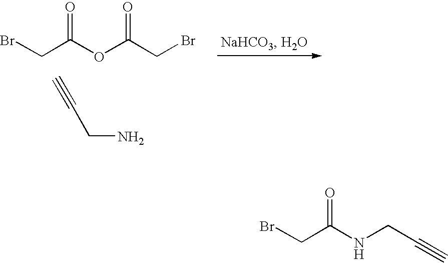

Propargylamine (0.1 ml, 1.45 mmol) was dissolved in water (15.0 ml) and was treated with bromoacetic anhydride (1.85 g, 7.25 mmol) in the presence of NaHCO₃ (2.5 g). The reaction was stirred at room temperature for 3 h. The reaction was quenched with HCl 5% (50 ml) and the product was extracted with ethylacetate (3×50 ml). The organic phase wash washed with 1 M NaOH (5×100 ml) and water (2×100 ml). The organic phase was dried with MgSO₄, filtered and concentrated under reduced pressure to afford a white crystalline solid (0.11 g, 43%). R_{f}=0.33 (petroleum ether/ethyl acetate 1:1). ¹H-NMR (250 MHz, CDCl₃) δ (ppm): 6.78 (1H, br, NH); 4.07 (2H, q, J=5.4 Hz, J=2.6 Hz, CH₂); 3.88 (2H, s, COCH₂Br); 2.27 (1H, t, J=2.6 Hz, CH). ¹³C-NMR (63 MHz, CDCl₃) δ (ppm): 165.3 (qC, CO); 78.5 (qC, alkyne); 72.2 (CH), 29.9 and 28.6 (CH₂). FAB-MS calculated for C₅H₆BrNO (M+1) 174.96. found: 197.85 and 199.85 (M+23).

### 1-N-(3,4,6-tri-O-acetyl-2-deoxy-2-N-acetyl-β-D-glucopyranosylamide)-4-(N′-methylidenyl-2′-bromoacetamido)-4,5-anhydro-triazole (5)

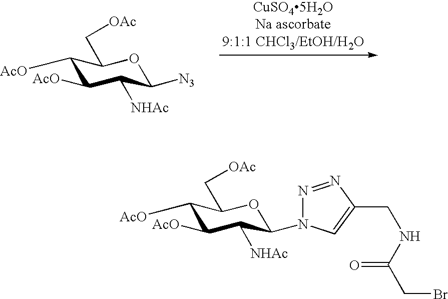

The azido sugar (100 mg, 0.27 mmol) and propargyl bromoacetamide (47 mg, 0.27 mmol) were dissolved in a biphasic solution of CHCl₃/EtOH/H₂O (9:1:1) (1.1 mL). Sodium ascorbate (54 mg, 0.27 mmol) and CuSO₄.5H₂O (2 mg, 0.007 mmol) were added. The reaction was stirred at 600 rpm, 50° C. overnight. The reaction mixture was then diluted with CHCl₃ and washed with saturated aqueous NaHCO₃ (3×20 mL), and the organic phase was dried with MgSO₄, filtered and concentrated under reduced pressure to afford a brown solid (98 mg, 66%). R_{f}: 0.40 (ethyl acetate). ¹H-NMR (300 MHz, CDCl₃ and 5% of CD₃OD) δ (ppm): 7.72 (1H, bp, CH-triazole); 5.76 (1H, d, J₁₋₂=9.7 Hz, H1); 5.21 (1H, dd, J₂₋₃=J₃₋₄=9.8 Hz, H3); 4.99 (1H, dd, J₃₋₄=J₄₋₅=9.8 Hz, H4); 4.28 (1H, bp, H2); 4.19 (2H, s, COCH₂Br); 4.08 (2H, dd, J_{6a-6b}=12.7 Hz, J₅₋₆ₐ=4.8 Hz, H6a); 3.92 (1H, dd, J_{6a-6b}=12.7 Hz, J_{5-6b}=1.8 Hz, H6b); 3.87-3.83 (1H, m, H5); 3.63 (2H, s, CH₂NH); 1.86, 1.85, 1.82 (9H, 3×s, CH₃CO); 1.52 (3H, s, CH₃CONH). ¹³C-NMR (75 MHz, CDCl₃ and 5% of CD₃OD) δ (ppm): 171.7, 170.9, 170.4, 169.6 (5×qC, CO); 82.0, 74.5, 72.0, 68.0, 53.1 (5×CH; C1-C5); 61.7 (CH₂, C6); 35.0 (CH₂, Ar—CH₂—NH); 28.0 (CH₂, CO—CH₂—Br); 21.8, 20.2, 20.1. 20.1 (4×COCH₃). FAB-MS calculated for C₁₉H₂₆BrN₅O₉ (M+1): 548.09867. found: 548.10034. NOTE: the lower yield stated here (compared to that quoted in table 1) can be attributed to the difficulties associated with the solubility of 5 during column chromatography

### N-propargyl-(2-thiobenzyl)acetamide (8)

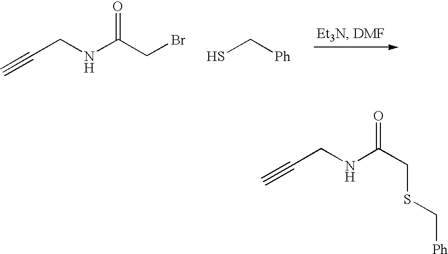

N-(propargyl)-bromacetamide (100 mg, 0.57 mmol) was dissolved in DMF (4.0 ml). Benzylmercaptan (700 μL, 5.77 mmol) and triethylamine (885 μL, 6.35 mmol) was added. The reaction was stirred for 16 h. The reaction mixture was diluted with chloroform (10.0 ml), and washed with a NaOH 1M (10.0 ml), 5% HCl (10.0 ml), saturated aqueous NaHCO₃ (10.0 ml) and water (10 ml). The organic phase was dried with MgSO₄, filtered and concentrated under reduced pressure. The crude product was purified by flash chromatography over silica (petroleum ether/ethyl acetate, 1:1) to afford the pure product (105 mg, 84%). R_{f}: 0.38 (petroleum ether/ethyl acetate 1:1). ¹H-NMR (300 MHz, CDCl₃) δ (ppm): 7.34-7.22 (5H, m, Ph); 6.91 (1H, bp, NH); 3.95 (2H, q, J=5.4 Hz, J=2.5 Hz, CH₂); 3.72 (2H, s, COCH₂S); 3.12 (2H, S, PhCH₂S); 2.24 (1H, t, J=2.5 Hz, CH). ¹³C-NMR (75 MHz, CDCl₃) δ (ppm): 168.4 (qC, CO); 137.0 (qC, Ph); 129.0, 128.8, 127.5 (5×CH, Ph) 79.3 (qC, alkyne); 71.8 (CH), 37.1, 35.0 and 29.4 (CH₂). FAB-MS calculated for C₁₂H₁₃NOS (M+1) 220.07906. found. 220.07910.

### 1-N-(3,4,6-tri-O-acetyl-2-deoxy-2-N-acetyl-β-D-glucopyranosylamido)-4-(N′-methylidenyl-2′-thiobenzylacetamido)-4,5-anhydro-triazole (9)

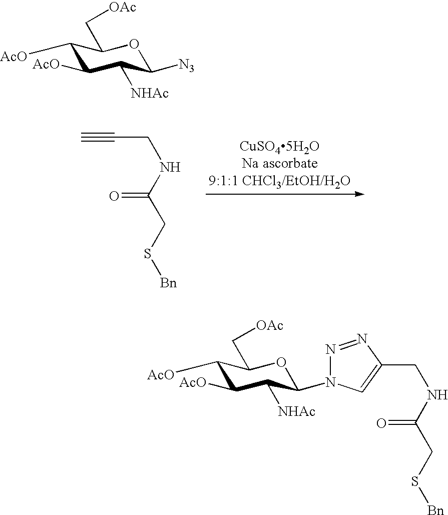

The azido sugar (100 mg, 0.27 mmol) and propargyl derivative (59 mg, 0.27 mmol) were dissolved in a biphasic solution of CHCl₃/EtOH/H₂O (9:1:1) (1.1 mL). Sodium ascorbate (54 mg, 0.27 mmol) and CuSO₄.5H₂O (2 mg, 0.007 mmol) were added. The reaction was stirred at 600 rpm, 50° C. overnight. Afterwards, the reaction mixture was diluted with CHCl₃ and washed with saturated aqueous NaHCO₃ (3×20 mL), and the organic phase was dried with MgSO₄, filtered and concentrated under reduced pressure to afford a pale brown solid (146 mg, 91%). R_{f}: 0.33 (ethyl acetate). ¹H-NMR (300 MHz, CDCl₃ and 5% of CD₃OD) δ (ppm): 7.75 (1H, s, CH-triazole); 7.24-7.18 (5H, m, Ph); 5.86 (1H, d, J₁₋₂=9.9 Hz, H1); 5.33 (1H, dd, J₂₋₃=J₃₋₄=9.9 Hz, H3); 5.12 (1H, dd, J₃₋₄=J₄₋₅=9.9 Hz, H4); 4.38 (1H, dd, J₁₋₂=J₂₋₃=9.9 Hz, H2); 4.35 (2H, s, CH₂NH); 4.19 (1H, dd, J_{6a-6b}=12.6 Hz, J₅₋₆ₐ=4.8 Hz, H6a); 4.03 (1H, dd, J_{6a-6b}=12.6 Hz, J_{5-6b}=1.9 Hz, H6b); 3.94 (1H, ddd, J₄₋₅=9.9 Hz, J₅₋₆ₐ=4.8 Hz, J_{5-6b}=1.9 Hz, H5); 3.66 (2H, s, COCH₂S); 3.03 (2H, s, SCH₂Ph); 1.97, 1.95, 1.93 (9H, 3×s, CH₃CO); 1.62 (3H, s, CH₃CONH). ¹³C-NMR (75 MHz, CDCl₃ and 5% of CD₃OD) δ (ppm): 171.4, 170.9, 170.6, 169.7, 169.6 (5×qC, CO); 137.1 (qC, triazole); 128.9, 128.5, 127.2 (CH, Ph); 121.2 (qC, Ph); 85.9, 74.7, 72.2, 68.0, 53.3 (5×CH; C1-C5); 61.7 (CH₂, C6); 36.8, 34.8, 34.7 (CH₂); 23.6, 22.2, 20.5, 20.4 (4×COCH₂). FAB-MS calculated for C₂₆H₃₃N₅O₉S (M+1): 592.20717. found: 592.20858.

### S-Benzyl thioether (9)

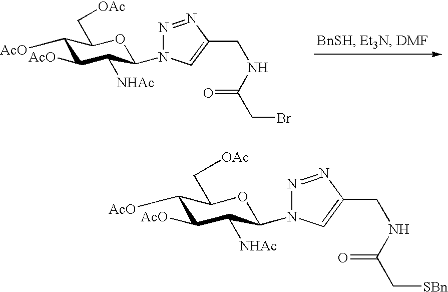

The sugar (50 mg, 0.09 mmol) was dissolved in DMF (615 μL). Benzylmercaptan (106 μL, 0.9 mmol) and triethylamine (138 μL, 0.726 mmol) was added. The reaction was stirred for 16 h. The reaction mixture was diluted with chloroform (10 mL), and washed with a NaOH (1 M, 10 mL), 5% HCl (10 mL), saturated aqueous NaHCO₃ (10 mL) and water (10 mL). The organic phase was dried with MgSO₄, filtered and concentrated under reduced pressure. The crude product was purified by flash chromatography over silica (petroleum ether/ethyl acetate, 9:1→1:9) to afford the pure product (40 mg, 75%). R_{f}: 0.33 (ethyl acetate). ¹H-NMR data as above.

### Deacetylated S-Benzyl thioether (10)

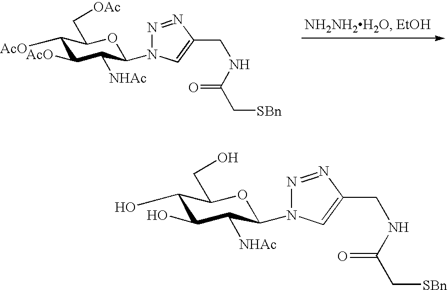

The sugar (137 mg, 0.23 mmol) was dissolved in a 2% solution of hydrazine monohydrate in ethanol (5 mL). After 3 days, the reaction was complete. The solvent was removed under high vacuum, and the crude product was purified by flash chromatography over silica (10% methanol in DCM) to afford the pure product (71 mg, 66%). R_{f} 0.01 (10% methanol in DCM). ¹H-NMR (300 MHz, D₂O/CD₃OD) δ (ppm): 8.04 (1H, s, CH-triazole); 7.30-7.24 (5H, m, Ph); 5.76 (1H, d, J₁₋₂=9.8 Hz, H1); 4.40 (2H, s, triazole-CH₂—NH); 4.20 (1H, dd, J₁₋₂=J₂₋₃=9.8 Hz, H2); 3.90-3.54 (5H, m, H3, H4, H5, H6a, H6b); 3.78 (2H, s, CO—CH₂—S); 3.12 (2H, s, S—CH₂-Ph); 1.76 (3H, s, COCH₃). ¹³C-NMR (75 MHz, D₂O/CD₃OD) δ (ppm): 173.5, 172.2 (2×qC, CO); 146.0, 139.0 (2×qC, Ph and triazole); 130.2, 129.5, 128.2, 123.0 (4×CH, Ph and triazole); 88.2, 81.2, 75.6, 71.4, 56.8 (5×CH; C1-C5); 62.3 (CH₂, C6); 37.5, 35.8, 35.5 (3×CH₂); 22.6 (COCH₃). FAB-MS calculated for C₂₀H₂₇N₅O₆S (M+1): 466.17548. found: 466.17335.

### Peptide Synthesis

Peptide synthesis was carried out using Rink amide-MBHA resin for the production of peptide thioesters (loading=0.67 mmol/g). All resins and Fmoc amino acids were purchased from Novabiochem. Mass spectra were obtained on a Micromass Quattro LC series electrospray mass spectrometer. Semi-preparative HPLC was performed using a Phenomenex LUNA C₁₈ column and a gradient of 5-95% acetonitrile containing 0.1% TFA over 45 minutes (flow rate of 3.0 mL/min). All other chemical reagents were obtained form Aldrich.

### Peptide Thioester Synthesis (EPO Residues 1-19).

The peptide thioesters were prepared using the dual linker strategy recently described by the Unverzagt group.¹

Briefly, rink amide resin (0.1 mmol) was deprotected by exposure to 20% piperidine in DMF. Fmoc-Phe-OH (5 equiv) was coupled using HBTU/HOBt as coupling reagents. The coupling time was 4 h. After Fmoc removal with 20% piperidine in DMF the sulfonamide linker was coupled through exposure of the resin to 3-carboxypropanesulfonic acid (50 mg, 0.3 mmol), HOBt (40 mg, 0.3 mmol) and DIC (47 μL, 0.3 mmol) for 5 h. The first amino acid (Fmoc-Ser(tBu)-OH, 5 equivalents per coupling) was then double coupled employing N-methylimidazole (40 μL, 0.5 mmol), DIC (78 μL, 0.5 mmol) as coupling reagents in 4:1 DCM/DMF for 16 h. The peptide was extended (target sequence: APPRLICDSRVLERYLLEA-SBn) and cleaved with benzylmercaptan, after ICH₂CN activation, using well established procedures.² The crude fully deprotected and precipitated peptide was redissolved in 25% aqueous MeCN and purified by semi-prep HPLC. The major peak (retention time=29 mins) was analysed by ESI-MS and was found to correspond to the desired product. This fraction was lyophilized to obtain approximately 1 mg of the product that was used in subsequent NCL reactions. NOTE: use of the double linker strategy indicated that although the resin activation with ICH₂CN had been near quantitative the subsequent release of the thioester was particularly sluggish as significant quantities of the activated resin-bound peptide remained attached to the solid support. Further peptide could be released by re-exposure of the resin to benzyl mercaptan and by conducting the cleavage reaction at 40° C.

Reaction of Bromoacetamides 4-7 with Solid-Supported Peptide 11

After MS verification that the desired peptide had been prepared the StBu protecting groups were cleaved on solid-phase by exposure to fresh 10% w/v dithiothreitol in dry DMF containing 2.5% v/v DIPEA for 2×24 h . The thiols were capped by treatment with the desired bromoacetamide (3 equivalents per thiol) in DMF containing 2.5% pyridine (or Et₃N) for 24 h.

### Native Chemical Ligation:

The NCL reaction was conducted under standard conditions. The peptides (1 mg each thioester and purified 13) were dissolved in 250 μL of 6M guanidine HCl, containing 300 mM Na phosphate buffer; pH 8.0, 1% w/v MESNA and 10 mM TCEP. The reaction was incubated at room temperature for 36 h. and loaded directly onto a semi-prep HPLC column. The HPLC showed a single major species with a retention time of 25.8 mins which was confirmed as the desired product by ESI-MS (calculated Mwt=5125.6 Da, Obs MWt=5127.0 Da.

Finally, the click reaction also works when acetylenes are loaded first onto solid phase (see reaction shown below). The resin (42 mg) containing the peptide shown in scheme 1 (9.16×10⁻⁶ mol peptide modified with 4 as described in the manuscript) was suspended in 9:1:1 CHCl₃/EtOH/50 mM sodium phosphate buffer (1.1 ml) and 1 (20 mg, 0.055×10⁻³ mol, 3 equiv per thiol) and sodium ascorbate (7 mg, 0.055×10⁻³ mol) were added followed by Cu(SO₄).5H₂O (0.5 mg). The reaction was incubated at 37° C. with shaking at 500 rpm in a 1.5 ml eppendorf tube, in an eppendorf thermomixer. The resin was then filtered and washed with water, NMP, and then DCM. The product was cleaved from the solid support by exposure to 95% TFA, 2.5% water, 2.5% EDT for 3 h and analyzed by mass spectrometry.

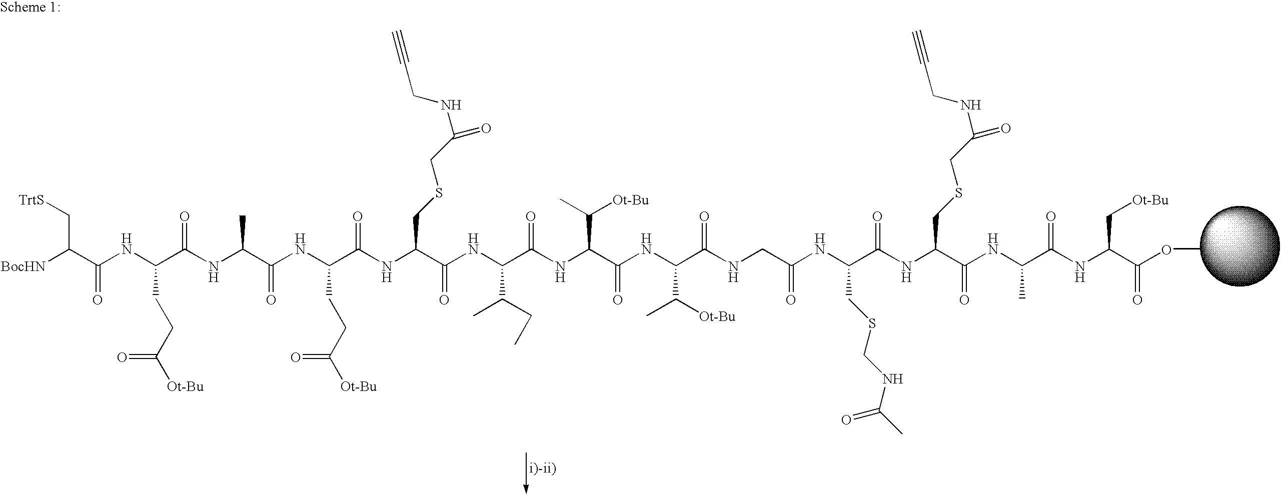

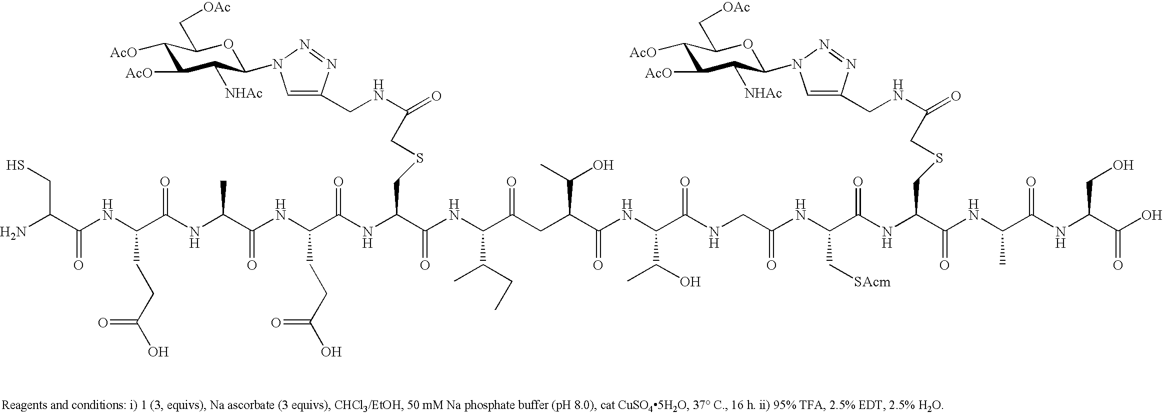

### Example 2

The present inventors also constructed larger polysaccharide compounds (the final products in the reactions shown in FIGS. 4 and 5, Compounds A and B, respectively) for attachment to a peptide chain, this polysaccharide having two di saccharide groups attached. A diagrammatic reaction scheme showing the synthesis of these compounds can be found in FIGS. 4 and 5.

### Experimental Details for Example 2

### General Techniques

### Instrumentation:

¹H NMR spectra were recorded at 250 and 300 MHz, ¹³C NMR spectra were recorded at 63 and 75 MHz and ¹⁹F NMR spectra were recorded at 235 MHz on a Bruker 250Y instrument. Chemical shifts (δ) were reported in ppm and coupling constants (J) in Hz, signals were sharp unless stated as broad (br), s: singlet, d: doublet, t: triplet, m: multiplet and q: quaternary. Residual protic solvent, CDCl₃ (δ_{H}: 7.26, s) was used as the internal standard in ¹H-NMR spectra unless otherwise stated. Electrospray mass spectroscopy was carried out on a Micromass Quattro LC electrospray with an applied voltage of 25-60V.

### Chromatography

Analytical TLC was carried out on Merck aluminium backed plates coated with silica gel 60F₂₅₄. Flash chromatography was carried out over Fisher silica gel 60 Å particle size 35-70 micron. Components were visualized using p-anisaldehyde dip and UV light (254 nm).

### Solvent and Reagents

All reagents and solvents were standard laboratory grade and used as supplied unless otherwise stated. Where a solvent was described as dry it was purchased as anhydrous grade. All organic extracts were dried over anhydrous magnesium sulphate prior to evaporation under reduced pressure.

### 2-Phthalamido-2-deoxy-1,3,4,6-tetra-O-acetyl-β-D-glucopyranose

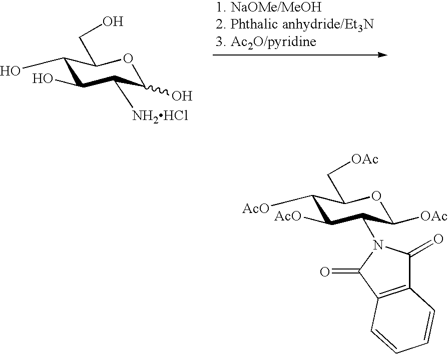

To a stirred solution of sodium methoxide (3.00 g, 46.3 mmol) in anhydrous methanol (75 ml) was added glucosamine hydrochloride (10.00 g, 46.3 mmol). The reaction mixture was stirred for 30 min at room temperature and then filtered with suction. Phthalic anhydride (3.50 g, 23.0 mmol) was then added to the filtrate and stirring was continued for further 20 min. A further portion of phthalic anhydride (3.50 g, 23 mmol) was then added followed by triethylamine (7.6 ml, 55.6 mmol). The reaction mixture was stirred at room temperature for 10 min and then cooled for 1 h in an ice bath and then filtered with suction. The precipitate was washed with cold methanol (2×20 ml) and dried under high vacuum. The dry white solid was then suspended in acetic anhydride (44.5 ml) and cooled down to 0° C., and then pyridine (22.7 ml) was added carefully with stirring. The reaction was the stirred at room temperature for 16 h. After this time, the reaction mixture was poured into ice/water (200 ml) and extracted with chloroform (3×200 ml). The combined organic extracts were washed with 5% HCl (1×120 ml), saturated aqueous NaHCO₃ (120 ml), water (120 ml) and brine (100 ml). The organic phase was dried MgSO₄, filtered and concentrated under reduced pressure to afford an orange oil. The crude product was purified by flash chromatography over silica (1:1 hexane/ethyl acetate) to afford the pure product (6.62 g; 30%). R_{f}: 0.56 (4:1 ethyl acetate/petroleum ether). ¹H-NMR (250 MHz, CDCl₃): δ (ppm): 7.74-7.62 (4H, m, ArH); 6.35 (1H, d, J₁₋₂=8.9 Hz, H-1); 5.73 (1H, dd, ³J₂₋₃=³J₃₋₄=9.8 Hz, H-3); 5.05 (1H, dd, ³J₃₋₄=³J₄₋₅=9.8 Hz, H-4); 4.34 (1H, dd, ²J_{6a-6b}=11.6 Hz, ³J₅₋₆ₐ=3.3 Hz, H-6a); 4.30 (1H, dd, ³J₁₋₂=8.9 Hz, ³J₂₋₃=9.8 Hz, H-2); 3.98 (1H, dd, ²J_{6a-6b}=11.6 Hz, ³J_{5-6b}=4.2 Hz, H-6b); 3.91 (1H, ddd, ³J₄₋₅=9.8 Hz, ³J₅₋₆ₐ=3.3 Hz, ³J_{5-6b}=4.2 Hz, H5); 1.92, 1.88, 1.81 and 1.70 (12H, 4×s, CH₃CO). ¹³C-NMR (63 MHz, D₂O) δ (ppm): 169.8, 169.3, 168.8, 167.9, 166.7 (qC, CO); 134.0 (Ar-4,7); 130.6 (Ar-3a,7a); 123.2 (Ar-5,6); 89.1, 72.0, 69.8, 67.7, 52.9 (5×CH, C1-C5); 60.9 (CH₂); 20.0 (COCH₃). FAB-MS calculated for C₂₂H₂₃NO₁₁: 476.9. found 494.9 [MNH₄]⁺.

### 3,4,6-Tri-O-acetyl-2-deoxy-2-phthalimido-β-D-glucopyranosyl azide

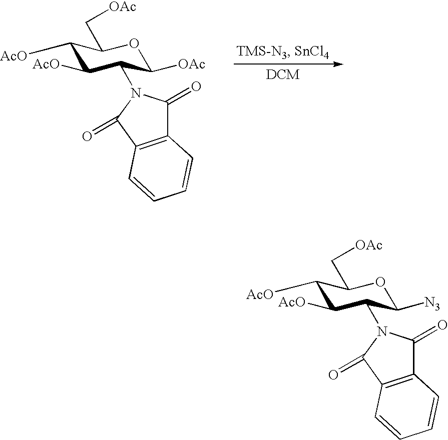

To a suspension of 1,3,4,6-Tetra-O-acetyl-2-deoxy-2-phthalimido-β-D-glucopyranose (2.36 g, 4.82 mmol) in DCM (25 ml), were added trimethylsilyl azide (0.77 ml, 5.78 mmol) and tin tetrachloride (0.28 ml, 2.41 mmol) and the mixture was stirred for 24 h. After that time TLC (3:1 ethyl acetate/petroleum ether) indicated that the reaction was complete. The reaction was diluted with DCM (50 ml) and washed with saturated aqueous NaHCO₃ (40 ml) and water (40 ml). The organic phase was dried MgSO₄, filtered and concentrated under reduced pressure. The crude product was dissolved in the minimum volume of DCM and saturated with methanol to afford the production of white crystals (1.612 g, 73%). R_{f}: 0.36 (3:1 ethyl acetate/petroleum ether). ¹H-NMR (250 MHz, CDCl₃): δ (ppm): 7.88-7.84 (2H, q, ³J₄₋₅=³J₆₋₇=5.5 Hz, ⁴J₄₋₆=⁴J₅₋₇=3.1 Hz, ArH-4, -7); 7.76-7.73 (2H, q, ³J₅₋₆=5.4 Hz, ⁴J₄₋₆=⁴J₅₋₇=3.1 Hz, ArH-5, -6); 5.79 (1H, dd, ³J₂₋₃=10.6 Hz, ³J₃₋₄=9.2 Hz, H-3); 5.64 (1H, d, ³J₁₋₂=9.5 Hz, H-1); 5.18 (1H, dd, ³J₃₋₄=³J₃₋₄=9.5 Hz, H-4); 4.38-4.16 (3H, m, H-2, H-6a, H-6b); 3.97 (1H, m, H5); 2.12, 2.03 and 1.85 (9H, 3×s, CH₃CO). ¹³C-NMR (63 MHz, D₂O) δ (ppm): 170.6, 170.0 and 169.4 (qC, CO); 134.5 (Ar-4,7); 131.2 (Ar-3a,7a); 123.7 (Ar-5,6); 85.5, 73.9, 70.3, 68.4, 53.9 (5×CH, C1-C5); 61.7 (CH₂); 20.7, 20.5 and 20.3 (COCH₂). FAB-MS calculated for C₂₀H₂₀N₄O₉ (M+NH₄⁺) 478.1230. found 478.1348.

### 6-O-tert-butyldiphenylsilyl-2-deoxy-2-phthalimido-β-D-glucopyranosyl azide

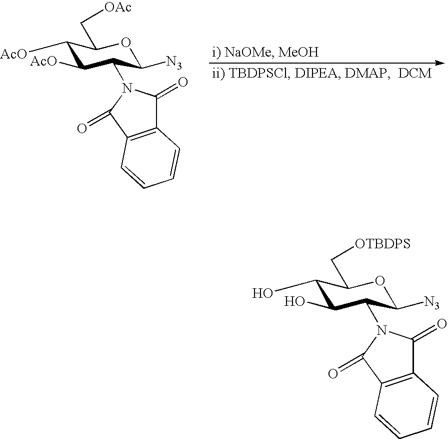

The glycosyl azide (1.612 g, 3.5 mmol) was dissolved in methanol (24 ml). A 0.5 M solution of NaOMe in methanol was added until a pH of 10 was obtained. After the reaction was stirred for 16 h, it was neutralized by addition of acetic acid. The mixture was then concentrated and azeotroped with toluene before being placed under high vacuum for 1 h. The resulting white solid was dissolved in DCM (12 ml) to which was added DIPEA (1.22 ml, 7.0 mmol) and DMAP (43 mg, 0.35 mmol). TBDPSCl (1.0 ml, 3.85 mmol) was then added, and the reaction was stirred for 16 h. After that time TLC (4:1 ethyl acetate/petroleum ether) indicated that the reaction was complete. The crude was washed with water (2×20 ml) and brine (2×20 ml). The organic phase was dried MgSO₄, filtered and concentrated under reduced pressure. The crude product was purified by flash chromatography over silica (5% methanol in DCM) to afford the pure product (1.868 g; 93%). R_{f}: 0.56 (4:1 ethyl acetate/petroleum ether). ¹H-NMR (250 MHz, CDCl₃): δ (ppm): 7.76-7.61 (9H, m, ArH); 7.43 (5H, m, ArH); 5.35 (1H, d, ³J₁₋₂=9.5 Hz, H-1); 4.40 (1H, dd, ³J₃₋₄=10.5 Hz, ³J₂₋₃=7.8 Hz, H-3); 4.05 (1H, dd, ³J₁₋₂=9.5 Hz, ³J₂₋₃=7.8 Hz, H-2); 3.97 (1H, m, H-5); 3.70 (1H, m, H-4); 3.65 (2H, m, H-6a, H-6b); 1.09 (9H, s, C(CH₃)₃). ¹³C-NMR (63 MHz, D₂O) δ (ppm): 168.2 (qC, CO); 135.5 (4×CH, C₆H₅); 134.2 (2×qC, Pht); 132.8 (2×CH, Pht); 131.3 (2×qC, C₆H₅); 129.8 (4×CH, C₆H₅); 127.7 (2×CH, C₆H₅); 123.5 (2×CH, Pht); 85.3, 77.2, 72.5, 71.2, 55.8 (5×CH, C1-C5); 63.8 (CH₂); 26.7 (3×CH₃, C(CH₃)₃); 19.1 (qC, C(CH₃)₃). FAB-MS calculated: 573.2091. found 573.2169.

### 1,2,3,4,6-penta-O-acetyl-β-D-galactopyranose

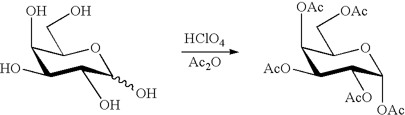

HClO₄ (0.1 ml) was added dropwise a solution of acetic anhydride (225 ml) at 0° C. and stirred for 1 h. Then, galactose (25.0 g, 0.130 mol) was added in small portions to the reaction mixture. After 3 h, TLC (7:3 ethyl acetate/hexane) indicated that the reaction was complete. The crude product was concentrated to one third of its original volume. The mixture was diluted with chloroform (600 ml) and washed with saturated aqueous NaHCO₃ (4×150 ml), water (150 ml) and brine (150 ml). The organic phase was dried with MgSO₄, filtered and evaporated under reduced pressure to afford a brown oil (54 g; 100%). R_{f}: 0.42 (7:3 ethyl acetate/hexane). ¹H-NMR (250 MHz, CDCl₃) δ (ppm): 6.34 (1H, d, ³J₁₋₂=1.7 Hz, H1); 5.46 (1H, d, ³J₄₋₅=1.1 Hz, H4); 5.39-5.19 (2H, m, H2, H3); 4.31 (1H, dd, ³J₅₋₆=6.5 Hz, ³J₄₋₅=1.1 Hz, H5); 4.05 (2H, dd, H6ₐ, H6_{b}); 2.18, 2.12, 2.00, 1.98 and 1.97 (15H, 5×s, CH₂CO). ¹³C-NMR (63 MHz, CDCl₃) δ (ppm): 170.3, 170.0, 168.9, 166.3 (5×qC, 5 CH₃CO); 89.6, 67.3, 67.2, 66.4 (5×CH, C1-C5); 61.2 (CH₂); 20.5 (5×CH₃, CH₃CO). FAB-MS calculated for C₁₆H₂₂O₁₁ (M+1) 391.3393. found 391.12404.

### 2,3,4,6-tetra-O-acetyl-D-galactose

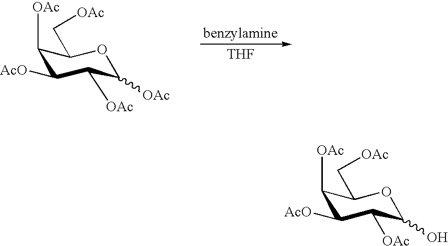

The peracetylated galactose (7.8 g, 20.0 mmol) was dissolved in anhydrous THF (120 ml) and benzylamine (2.6 ml, 24.0 mmol) was added under nitrogen. The reaction mixture was then stirred at 50° C. for 24 h. After that time TLC (4:1 ethyl acetate/petroleum ether) indicated the presence of both hemi-acetal and some unreacted starting material. Then the reaction mixture was concentrated under reduced pressure. The crude product was purified by flash chromatography over silica (4:1 ethyl acetate/petroleum ether) to afford the purified product (6.24 g; 95%). R_{f}: 0.46, 0.30 (4:1 ethyl acetate/petroleum ether). ¹H-NMR (250 MHz, CDCl₃), ¹³C-NMR (63 MHz, CDCl₃): anomeric α:β mixture. FAB-MS calculated for C₁₄H₂₀O₁₀: 348.3. found: 371.3 [MNa]⁺.

### 2,3,4,6-tetra-O-acetyl-D-galactopyranosyl trichloroacetimidate

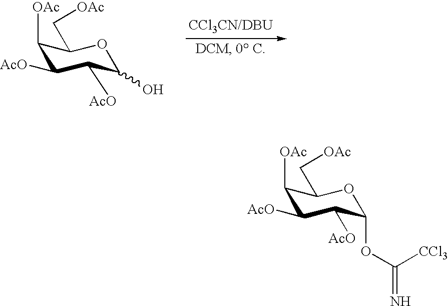

A solution of the monosaccharide (100 mg, 0.29 mmol) in anhydrous DCM (1.86 ml), was cooled to 0° C., and trichloroacetonitrile (872.0 μL, 8.7 mmol) and DBU (43 μL, 0.29 mmol) were added. After 2 h stirring at 0° C. under N₂, the reaction was concentrated to afford a solid. The crude product was purified by flash chromatography over silica (1:1 ethyl acetate/petroleum ether) to afford the purified product (0.129 g; 90%), as a brown oil. R_{f}: 0.34 (2:1 ethyl acetate/petroleum ether). ¹H-NMR (250 MHz, CDCl₃) δ (ppm): 8.65 (1H, s, NH); 6.59 (1H, d, ³J₁₋₂=3.2 Hz, H1); 5.55 (1H, dd, ³J₃₋₄=2.9 Hz, ³J₄₋₅=1.0 Hz, H4); 5.42 (1H, dd, ³J₂₋₃=10.8 Hz, ³J₃₋₄=2.9 Hz, H3); 5.35 (1H, dd, ³J₂₋₃=10.8 Hz, ³J₁₋₂=3.2 Hz, H2); 4.43 (1H, dt, ³J₅₋₆=7.2 Hz, ³J₄₋₅=1.0 Hz, H5); 4.16 (1H, dd, ²J_{6a-6b}=11.2 Hz, ³J₅₋₆ₐ=7.2 Hz, H6ₐ); 4.07 (1H, dd, ²J_{6a-6b}=11.2 Hz, ³J_{5-6b}=6.8 Hz, H6_{b}); 2.16-2.00 (12H, s, CH₃CO). ¹³C-NMR (63 MHz, CDCl₂) δ (ppm): 170.7, 170.5, 170.4, 161.3 (6×qC, 4 CH₃CO, CNH, CCl₂); 93.9, 69.4, 67.9, 67.7, 67.3 (5×CH, C1-C5); 61.6 (CH₂); 21.0 (4×CH₃, 4 CH₃CO). FAB-MS calculated for C₂₆H₂₀NO₁₀Cl₃: 492.7. found: 515.7 [MNa]⁺.

### O-(2,3,4,6-Tetra-O-acetyl-β-D-galactopyranosyl)-(1→4)-6-O-tert-butyldiphenylsilyl-2-deoxy-2-phtalimido-β-D-glucopyranosyl azide

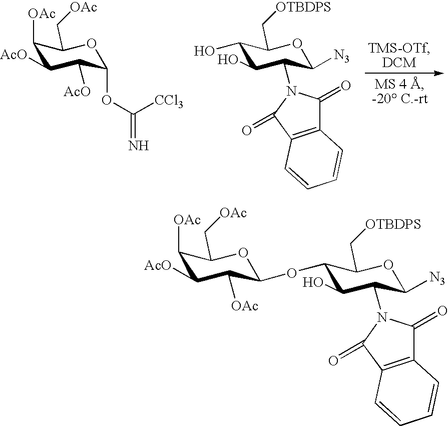

The trichloroacetimidate (4.69 g, 9.5 mmol) and the deprotected sugar (2.72 g, 4.75 mmol) were dissolved in dry DCM (25 ml), containing 4 Å molecular sieves, and the solution was cooled down to −20° C. A solution of 0.1 M TMS-OTf in dry DCM (4.75 ml, 0.475 mmol) was added. After 2 h, the reaction was warmed to RT. After 1 h, TLC (8:2 toluene/ethyl acetate) indicated that the reaction was finished. The reaction mixture was diluted with DCM (50 ml) and neutralized with solid NaHCO₃, then was filtered and the solvent was removed under reduced pressure. The crude product was purified by flash chromatography over silica (8:2 toluene/ethyl acetate) to afford the purified product (3.93 g, 92%) as a white foam. R_{f}: 0.26 (8:2 toluene/ethyl acetate). ¹H-NMR (250 MHz, CDCl₃): δ (ppm): 7.66 and 7.35 (10H, m, 2×C₆H₅); 7.16-7.06 (4H, m, Pht-H); 5.36 (1H, d, ³J_{1′-2′}=9.5 Hz, H-1′); 5.27 (1H, d, ³J₃₋₄=³J₄₋₅=3.3 Hz, H-4); 5.15 (1H, dd, ³J₂₋₃=10.4 Hz, ³J₁₋₂=8.1 Hz, H-2); 4.90 (1H, dd, ³J₂₋₃=10.4 Hz, ³J₃₋₄=3.3 Hz, H-3); 4.65 (1H, d, ³J₁₋₂=8.1 Hz, H-1); 4.42 (1H, dd, 10.4 Hz, ³J_{2′-3′}=10.4 Hz, ³J_{3′-4′}=8.5 Hz, H-3′); 4.06 (1H, dd, ³J_{2′-3′}=10.4 Hz, ³J_{1′-2′}=9.5, H-2′); 4.00-3.45 (7H, m, H5, H6a, H6b, H4′, H5′, H6a′, H6b′), 2.26, 2.04, 1.90, 1.65 (12H, 4×s, COCH₃); 1.04 (9H, s, C(CH₃)₃). ¹³C-NMR (63 MHz, D₂O) δ (ppm): 170.4, 170.0, 169.9, 169.2, 168.1, 167.5 (6×qC, CO and COCH₃); 135.4 (4×CH, C₆H₅); 134.0 (2×qC, Pht); 132.8 (2×CH, Pht); 131.3 (2×qC, C₆H₅); 129.7 (4×CH, C₆H₅); 127.7 (2×CH, C₆H₅); 123.4 (2×CH, Pht); 100.9, 85.2, 80.0, 77.2, 71.9, 71.1, 70.6, 68.6, 66.8, 55.8 (10×CH, C1-C5, C1′-C5′); 63.5, 61.2 (2×CH₂); 26.6 (3×CH₃, C(CH₃)₃); 20.3 (4×COCH₃); 19.1 (qC, C(CH₃)₃). FAB-MS calculated for C₄₄H₅₀N₄O₁₅Si (M+1) 903.3042. found 903.3117.

### 2,3,4,6-Tetra-O-acetyl-β-D-galactopyranosyl-(1→4)-3,6-di-O-acetyl-2-acetamido-2-deoxy-β-D-glucopyranosyl azide

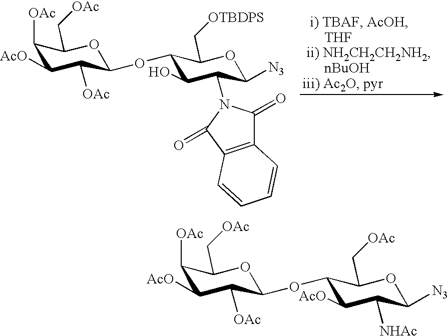

To a solution of the phthalimido disaccharide (946 mg, 1.05 mmol) and acetic acid (600 μL, 10.5 mmol) at 0° C. in THF (31.5 ml), was added a 1 M solution of TBAF in THF (4.2 ml, 4.2 mmol). The reaction mixture was stirred for 15 h at room temperature. The mixture was subsequently concentrated to a yellow oil. This oil was dissolved in n-BuOH (20 ml), and ethylendiamine (5 ml) was added. The reaction was heated to 90° C. and stirred for 16 h. The mixture was then concentrated under high vacuum and dissolved in a solution of 2:1 pyridine/Ac₂O (100 ml) and the reaction was stirred for 8 hours. The reaction was again concentrated to afford a yellow oil which was purified by silica gel chromatography (the column was packed with 1% methanol in DCM; the mobile phase was 3% methanol in DCM) to afford a purified product (1.18 g; 76%) as a white foam. R_{f}: 0.23 (3% methanol in DCM). ¹H-NMR (360 MHz, CDCl₃) δ (ppm): 5.96 (1H, d, ³J_{NH-2}=9.5 Hz, NHAc); 5.32 (1H, dd, ³J₃₋₄=3.3 Hz, H4); 5.10-5.04 (2H, m, H2, H1′); 4.96 (1H, dd, ³J₂₋₃=10.2 Hz, ³J₃₋₄=3.2 Hz, H3); 4.53-4.48 (2H, m, H1, H3′); 4.12-3.71 (7H, m, H5, H6a, H6b, H2′, H4′, H6a, H6b); 3.68 (1H, m, H5′); 2.26, 2.13, 2.10, 2.08, 2.04, 1.20, 1.95 (21H, 7×s, COCH₃). ¹³C-NMR (63 MHz, CDCl₃) δ (ppm): 171.0, 170.4, 170.3, 170.1, 170.0 and 169.3 (7×qC, CO); 101.1, 88.3, 75.6, 74.5, 72.6, 70.6, 70.5, 68.9, 66.4, 52.9 (10×CH, C1-C5, C1′-C5′); 61.7, 60.6 (2×CH₂); 23.0, 20.7, 20.5, 20.4 (7×COCH₃). FAB-MS calculated for C₂₆H₃₆N₄O₁₆ (M+1) 661.2126. found 661.1630.

### 2,3,4,6-Tetra-O-acetyl-β-D-galactopyranosyl-(1→4)-2-acetamido-2-deoxy-3,6-di-O-acetyl-1-N-[1-(2-bromo)acetyl]-β-D-glucopyranose

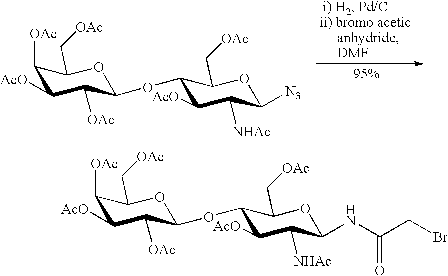

The disaccharide (1.98 g, 3.00 mmol) and 10% Pd/C (205 mg), were dissolved in anhydrous methanol (90 ml), and stirred at room temperature under an atmosphere of hydrogen for 2 h. The catalyst was then removed by filtration through Celite, and the Celite pad was washed with methanol. The solvent was removed under vacuum. The crude reaction was redissolved in anhydrous DMF (20 ml), and bromoacetic anhydride (1.01 g, 3.9 mmol) was added. The reaction was stirred at room temperature overnight. The reaction was diluted with chloroform (200 ml), and washed with 5% HCl (200 ml), saturated aqueous NaHCO₃ (200 ml), and water (200 ml). The organic phase was dried MgSO₄, filtered and concentrated under reduced pressure. The crude product was purified by flash chromatography over silica (2.5% methanol in DCM) to afford the pure product (2.19 g; 95%). R_{f}: 0.25 (2% methanol in DCM). ¹H-NMR (250 MHz, CDCl₃) δ (ppm): 7.65 (1H, d, ³J_{NH}=8.1 Hz, C1′-NHCO); 7.04 (1H, d, ³J_{NH}=8.3 Hz, NHAc); 5.27 (1H, dd, ³J₃₋₄=3.0 Hz, ³J₄₋₅=0.6 Hz, H-4); 5.05 (1H, m, H3′); 4.97 (1H, dd, ³J₁₋₂=³J₂₋₃=10.1 Hz, H2), 4.90 (1H, dd, ³J₂₋₃=10.1 Hz, ³J₃₋₄=3.2 Hz, H3); 4.42 (1H, d, ³J₁₋₂=10.1 Hz, H1); 4.35 (1H, d, ³J_{1′-2′}=11.61 Hz, H1′); 4.06-3.66 (8H, m, H2′, H4′, H5, H5′, H6a, H6b, H6a′, H6b′); 3.75 (2H, s, COCH₂Br); 2.11, 2.09, 2.08, 2.05, 2.04, 1.98, 1.94 (21H, 7×s, CH₃CO). ¹³C-NMR (63 MHz, D₂O) δ (ppm): 172.2, 171.0, 170.3, 170.1, 169.2 and 167.4 (8×qC, CO); 101.0, 80.0, 76.8, 74.4, 72.7, 70.9, 70.6, 68.9, 66.9, 52.7 (10×CH, C1-C5, C1′-C5′); 62.0, 60.9, 28.2 (3×CH₂); 22.9, 20.9, 20.6, 20.5 (7×COCH₃). FAB-MS calculated for C₂₈H₃₉BrN₂O₁₇ (M+1) 754.14. found 777.4 and 779.4.

### 2-Acetamido-2-deoxy-3,4,6-tri-O-acetyl-α-D-glucopyranosyl chloride

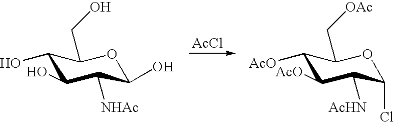

N-acetyl glucosamine (20 g, 90.46 mmol) was added to stirring acetyl chloride (30 ml). The suspension was stirred magnetically for 20 h. Afterwards the reaction was diluted with CHCl₃ (50 ml) and the resulting solution was washed with ice-water (50 ml) and ice-saturated aqueous NaHCO₃ (50 ml). The organic phase was dried MgSO₄, filtered and concentrated under reduced pressure. The crude product was purified by silica gel chromatography (the column was packed with 1:1 petroleum ether/ethyl acetate; mobile phase 1:2) to afford a purified product (11.36 g, 34%). R_{f}: 0.40 (Ethyl acetate). ¹H-NMR (300 MHz, CDCl₃) δ (ppm): 6.15 (1H, d, ³J₁₋₂=3.7 Hz, H1); 6.03 (1H, d, ³J_{NH-2}=8.7 Hz, NH); 5.30 (1H, dd, ³J₂₋₃=³J₃₋₄=9.9 Hz, H3); 5.17 (1H, ³J₃₋₄=³J₄₋₅=9.9 Hz, H4); 4.51 (1H, ddd, ³J₂₋₃=9.9 Hz, ³J_{NH-2}=8.7 Hz, ³J₁₋₂=3.7 Hz, H2); 4.28-4.20 (2H, m, H5, H6a), 4.11-4.07 (1H, m, H6b); 2.06, 2.01 and 1.95 (9H, s, CH₃CO). ¹³C-NMR (75 MHz, CDCl₃) δ (ppm): 171.4, 170.6, 170.2, 169.1 (4×qC, CO); 93.7, 70.9, 70.1, 67.0, 53.4 (C1-C5); 61.1 (CH₂); 23.0, 20.7, 20.5 (4×COCH₃). FAB-MS calculated for C₁₄H₂₀NO₈Cl (M+1) 366, found 366.

### 2-Acetamido-2-deoxy-3,4,6-tri-O-acetyl-α-D-glucopyranosyl azide

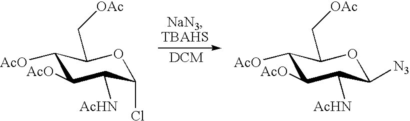

To a solution of the glucosyl chloride (11.36 g, 31.12 mmol), TBAHS (10.57 g, 31.12 mmol) and NaN₃ (6.07 g, 93.36 mmol) in DCM (110 ml), was added saturated aqueous NaHCO₃ (110 ml). The resulting biphasic solution was stirred vigorously at room temperature for 1 h. Ethyl acetate (200 ml) was then added and the organic layer was separated and washed with saturated aqueous NaHCO₃ (100 ml), water (2×100 ml). The organic phase was dried MgSO₄, filtered and concentrated under reduced pressure, to afford the pure product (9.54 g, 82%). R_{f}: 0.30 (Ethyl acetate). ¹H-NMR (300 MHz, CDCl₃) δ (ppm): 6.02 (1H, d, ³J_{NH-2}=8.9 Hz, NH); 5.26 (1H, ³J₃₋₄=9.8 Hz, ³J₂₋₃=9.6 Hz, H3); 5.08 (1H, ³J₃₋₄=³J₄₋₅=9.8 Hz, H4); 4.78 (1H, d, ³J₁₋₂=9.3 Hz, H1); 4.25 (1H, dd, ²J_{6a-6b}=12.4 Hz, ³J₅₋₆ₐ=4.8 Hz, H6a); 4.14 (1H, dd, ²J_{6a-6b}=12.4 Hz, ³J_{5-6b}=2.3 Hz, H6b); 3.91 (1H, ddd, ³J₂₋₃=9.6 Hz, ³J₁₋₂=9.3 Hz, ³J_{NH-2}=8.9 Hz, H2); 3.80 (1H, ddd, ³J₄₋₅=9.8 Hz, ³J₅₋₆ₐ=4.8 Hz, ³J_{5-6b}=2.3 Hz, H5); 2.10, 2.02, 2.01 and 1.96 (12H, s, CH₃CO). ¹³C-NMR (75 MHz, CDCl₃) δ (ppm): 170.9, 170.7, 170.6, 169.3 (4×qC, CO); 88.4, 73.9, 72.2, 68.2, 54.1 (C1-C5); 61.9 (CH₂); 23.2, 20.7, 20.6, 20.6 (4×COCH₃). FAB-MS calculated for C₁₄H₂₀N₄O₈ (M+1) 372.3. found 373.

### 2-Acetamido-2-deoxy-4,6-O-p-methoxybenzylidene-β-D-glucopyranosyl azide

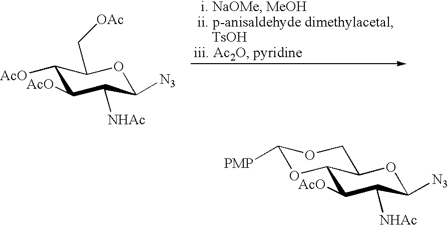

The azido monosaccharide (3 g, 8.07 mmol) was dissolved in dry MeOH (15 ml) and sodium methoxide (200 μL of a 0.5 M solution in methanol) was added and the reaction mixture was stirred for 2 h at room temperature. The reaction mixture was neutralized by adding acetic acid (10 μL), and concentrated under reduced pressure to afford the crude deacetylated product as a pale yellow foam. The crude azide was dissolved in anhydrous DMF (10 ml) and p-anisaldehyde dimethylacetal (3.37 g, 18.54 mmol) and p-Tosic acid (0.28 g, 1.61 mmol) were added. After stirring for 1.5 h at 50° C., the reaction mixture was concentrated under vacuum. The residue was poured into a cold mixture of saturated aqueous NaHCO₃ (30 ml) and DCM (30 ml) and cooled for 10 min at 4° C. The precipitate was crystallized from ethyl acetate (30 ml). The product was filtered, dried under vacuum and isolated as a white solid. The resulting product was dissolved in pyridine (20 ml) and acetic anhydride (10 ml). The mixture was stirred for 24 h at room temperature and then concentrated under vacuum. Dichloromethane (150 ml) was then added and the organic phase was washed with water (20 ml), saturated aqueous NaHCO₃ (20 ml). The organic layer was dried MgSO₄, and the solvent was removed under vacuum to afford the crude product as a white solid which was crystallised form ethyl acetate (1.19 g, 70%). R_{f}: 0.30 (Ethyl acetate). ¹H-NMR (300 MHz, CDCl₃) δ (ppm): 7.34 (2H, d, ³J=8.7 Hz, ArH); 6.88 (2H, d, ³J=8.8 Hz, ArH); 5.88 (1H, d, ³J_{H2-NHAc}=9.5 Hz, NHAc); 5.48 (1H, s, CHPh); 5.24 (1H, dd, ³J₂₋₃=9.9 Hz, H3); 4.48 (1H, d, ³J₁₋₂=9.2 Hz, H1); 4.32 (1H, dd, ²J_{6a-6b}=10.5 Hz, ³J₅₋₆ₐ=4.6 Hz, H6a); 4.11 (1H, ddd, ³J₂₋₃=9.9 Hz, ³J_{H2-NHAc}=9.5 Hz, ³J₁₋₂=9.2 Hz, H2); 3.79 (3H, s, CH₃O); 3.77-3.61 (3H, m, H4, H5, H6b); 2.09 and 2.00 (6H, s, CH₃CO). ¹³C-NMR (75 MHz, CDCl₃) δ (ppm): 171.4, 170.9, 159.8 and 128.8 (qC); 127.1 (2×ArCH); 113.2 (2×ArCH); 101.2, 88.8, 78.1, 71.4 and 68.1 (CH); 67.9 (CH₂); 54.9 (OCH₃); 53.3 (CH); 22.2 and 20.3 (2×COCH₃). FAB-MS calculated for C₁₈H₂₂N₄O₇ (M+Na) 429. found 429.

### 2-Acetamido-2-deoxy-3-acetyl-6-O-p-methoxybenzyl-β-D-glucopyranosyl azide

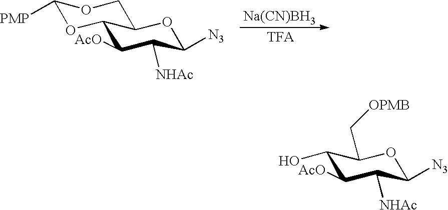

TFA (4.19 g, 36.8 mmol) in dry DMF (22.1 ml) was cooled down to 0° C., and was added dropwise to a stirring solution of the sugar azide (1.5 g, 3.68 mmol), sodium cyanoborohydride (1.16 g, 18.40 mmol) and 3 Å molecular sieves in dry DMF (29.4 ml) at 0° C. in an ice bath. After the addition was complete, the ice bath was removed and the reaction mixture was stirred at room temperature for 16 h. Next, the reaction was filtered with suction. The filtrate was poured into ice-cold saturated aqueous NaHCO₃ and the product was extracted with dichloromethane (5×60 ml). The combined organic extracts were washed with a saturated aqueous solution of NH₄Cl (100 ml) and water (100 ml). The organic phase was dried MgSO₄, filtered and concentrated under reduced pressure. The crude product was purified by silica gel chromatography (the column was packed with 1:1 petroleum ether/ethyl acetate; mobile phase: ethyl acetate) to afford a purified product (1 g, 66%). R_{f}: 0.20 (Ethyl acetate). ¹H-NMR (300 MHz, CDCl₃) δ (ppm): 7.28 (2H, d, ²J=8.7, ArH); 6.91 (2H, d, ²J=8.7, ArH); 6.02 (1H, d, ³J_{H2-NHAc}=9.2 Hz, NHAc); 5.08 (1H, dd, ³J₂₋₃=10.5 Hz, ³J₃₋₄=9.2 Hz, H3); 4.59 (1H, d, ³J₁₋₂=9.3 Hz, H1); 4.54 (2H, q, OCH₂Ph); 4.09 (1H, ddd, ³J₂₋₃=10.5 Hz, ³J₁₋₂=9.3 Hz, ³J_{H2-NHAc}=9.2 Hz; H2); 3.78 (3H, s, CH₃O); 3.77-3.46 (4H, m, H4, H5, H6a, H6b); 3.26 (1H, bp, OH); 2.09 and 1.96 (6H, s, CH₃CO). ¹³C-NMR (75 MHz, CDCl₃) δ (ppm): 171.8, 171.4 (CH₃CO); 159.3 (qC, Ar); 129.7 (qC, Ar); 129.4 (2×ArCH); 113.8 (2×ArCH); 88.8, 77.4, 75.4, 68.6 and 53.1 (C1-C5); 73.4 (OCH₂Ph); 68.9 (CH₂); 55.1 (OCH₃); 22.7 and 20.7 (2×COCH₃). FAB-MS calculated for C₁₈H₂₄N₄O₇ (M+Na) 431.4. found 431.4.

### 2-Acetamido-2-deoxy-3-acetyl-4-tert-butoxycarboxymethyl-6-O-p-methoxybenzyl-β-D-glucopyranosyl azide

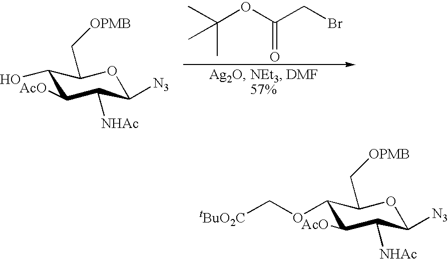

To a solution of the sugar derivative (2.28 g, 5.6 mmol) in DMF (15 ml) at 0° C., NEt₃ (1.6 ml, 11.2 mmol) was added and stirred for 30 min. Then, tert-butylbromo acetate (3 ml, 20.3 mmol) and silver oxide (2.3 g, 10.2 mmol) were added. The reaction was stirred overnight at room temperature under nitrogen gas with exclusion of light. The crude reaction was diluted with DCM (50 ml) and filtered through Celite. The organic phase was washed with saturated aqueous NaHCO₃ (4×50 ml) and water. The organic phase was dried MgSO₄, filtered and concentrated under reduced pressure. The crude product was purified by silica gel chromatography (the column was packed with 1:1 petroleum ether/ethyl acetate; mobile phase 1:2) to afford a purified product (1.67 g, 57%). Rf: 0.5 (ethyl acetate). ¹H-NMR (300 MHz, CDCl₃) δ (ppm): 7.21 (2H, d, ³J=8.7, ArH); 6.85 (2H, d, ³J=8.7, ArH); 6.35 (1H, d, ³J_{H2-NHAc}=9.6 Hz, NHAc); 5.14 (1H, dd, ³J₂₋₃=10.9 Hz, ³J₃₋₄=8.4 Hz, H3); 4.57 (1H, dd, ³J₃₋₄=³J₄₋₅=8.4 Hz, H4); 4.43 (1H, d, ³J₁₋₂=11.5 Hz, H1); 4.02 (1H, m, H2); 3.98 (2H, s, COCH₂Br); 3.77 (3H, s, CH₃O); 3.75-3.58 (3H, m, H5, H6a, H6b); 2.07 and 1.95 (6H, s, CH₃CO); 1.41 (9H, s, (CH₃)₃C). ¹³C-NMR (75 MHz, CDCl₃) δ (ppm): 171.4, 170.5, 168.6 (3×CO); 159.3 (qC, Ar); 130.1 (qC, Ar); 129.6, 129.4 (2×ArCH); 113.8 (2×ArCH); 88.7, 81.8, 76.7, 75.1 and 53.4 (C1-C5); 73.2, 70.3 and 67.9 (3×CH₂); 55.3 (OCH₃); 28.1 (3×CH₂); 22.7 and 20.7 (2×COCH₂). FAB-MS calculated for C₂₄H₃₄N₄O₉ (M+Na) 545.5. found 545.5.

### 2-Acetamido-2-deoxy-3,6-diacetyl-4-tert-butoxycarboxymethyl-β-D-glucopyranosyl azide

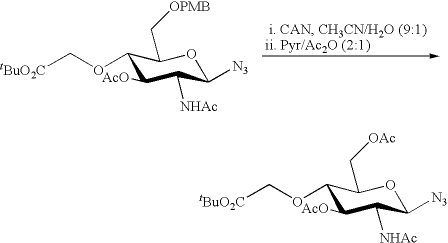

The azido sugar (145 mg, 0.28 mmol) was dissolved in 9:1 MeCN/H₂O (1.5 ml) and CAN (307 mg, 0.56 mmol) was added. After 1 h, TLC (ethyl acetate) indicated that the reaction was completed. The crude reaction was diluted with DCM (25 ml) and washed with saturated aqueous NaHCO₂ (10 ml). The organic phase was dried MgSO₄, filtered and concentrated under reduced pressure. The crude product was purified by silica gel chromatography (the column was packed with 1:1 petroleum ether/ethyl acetate; mobile phase 1:2) to afford a purified product (95 mg, 85%). R_{f}: 0.5 (ethyl acetate) ¹H-NMR (300 MHz, CDCl₃) δ (ppm): 6.32 (1H, d, ³J=9.6 Hz, NHAc); 5.17 (1H, dd, ³J₃₋₄=10.4 Hz, J₂₋₃=9.2 Hz, H3); 4.65 (1H, d, ³J₁₋₂=9.2 Hz, H1); 4.43 (1H, dd, ²J_{6a-6b}=12.2 Hz, ³J₅₋₆ₐ=2.1 Hz, H6a); 4.29 (1H, dd, ²J_{6a-6b}=12.2 Hz, ³J_{5-6b}=4.7 Hz, H6b); 4.12-3.96 (3H, m, H4, CH₂); 3.78 (1H, m, H5); 3.53 (1H, dd, ³J₁₋₂=³J₂₋₃=9.2 Hz, H2); 2.10, 2.08, 1.96 (9H, s, CH₃CO); 1.42 (9H, s, (CH₃)₃C). ¹³C-NMR (75 MHz, CDCl₃) δ (ppm): 171.3, 170.6, 170.4, 168.4 (4×qC, CO); 88.5, 77.5, 74.4, 70.2, 53.3 (5×CH, C1-C5); 82.1 (qC, C(CH₃)₃); 74.4, 62.7 (2×CH₂); 28.0 (3×CH₃, C(CH₃)₃); 23.1, 21.1, 20.8 (3×COCH₃). FAB-MS calculated for C₁₈H₂₈N₄O₉ (M+Na) 467.4, found 467.4.

### 2-Acetamido-2-deoxy-3,6-diacetyl-4-carboxymethyl-β-D-glucopyranosyl azide

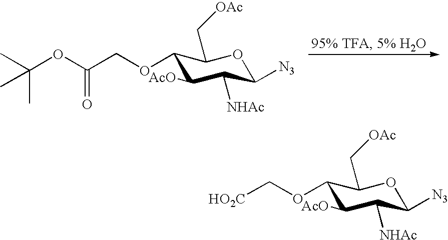

To a solution of the monosaccharide derivative (95 mg, 0.21 mmol) in TFA/H₂O (95:5, 5 ml), was stirred at room temperature for 2 h. Afterwards, the solution was concentrated to give the desired compound. (82 mg; 100%). ¹H-NMR (300 MHz, CDCl₃) δ (ppm): 6.67 (1H, d, ³J=9.4 Hz, NHAc); 5.21 (1H, dd, ³J₂₋₃=³J₃₋₄=9.5 Hz, H3); 4.68 (1H, d, ³J₁₋₂=9.4 Hz, H1); 4.49 (dd, ²J_{6a-6b}=11.6 Hz, H6a); 4.31 (dd, ²J_{6a-6b}=11.6 Hz , ³J_{5-6b}=4.4 Hz, H6b); 4.24 (2H, d, ³J=3.7 Hz, CH₂); 4.01 (1H, ddd, ³J=9.4 Hz, H2); 3.77 (1H, m, H5); 3.61 (1H, dd, ³J₃₋₄=³J₄₋₅=9.5 Hz, H4); 2.12, 2.09 and 2.03 (9H, s, CH₃CO). ¹³C-NMR (75 MHz, CDCl₃) δ (ppm): 171.2, 170.4, 170.1, 168.0 (4×qC, CO); 87.7, 75.1, 74.2, 70.1, 54.3 (5×CH, C1-C5); 75.2, 63.4 (2×CH₂); 22.2, 20.7 (3×COCH₃). FAB-MS calculated for C₁₄H₂₀N₄O₉ (M+1) 389.33. found 389.31 and (M+Na) 411.25.

### Synthesis of Compound A:

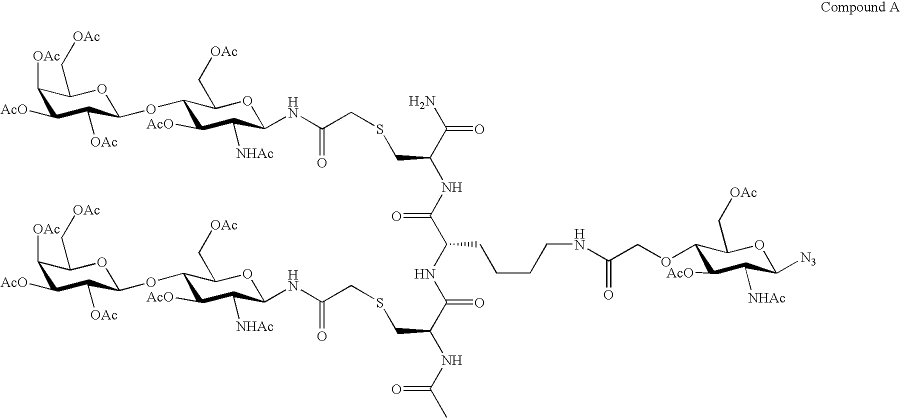

### General.

Solid-phase peptide synthesis was carried out manually using a plastic syringe fitter with a Teflon filter and connected to a vacuum waste chamber via a Teflon valve. Synthesis of the glycopeptide was carried out on MBHA Rink amide resin (loading=0.58 mmol/g).

### General Solid-Phase Peptide Synthesis.

The scale of the synthesis of the compound A was 0.05 mmol. The Fmoc group was removed by treatment with 20% piperidine in DMF (5 and 15 min) between each coupling and deprotection step. Coupling reactions with Fmoc amino acids were conducted using 0.25 mmol (5 equiv.) of each of HBTU/HOBt and DIPEA for 3 h. The reaction progress was monitored using the Kaiser ninhydrin test. Between each coupling and deprotection, the resin was washed with DCM and DMF (5 min each).

### General StBu Deprotection.

DTT (100 mg) was dissolved in dry DMF (0.9 ml) and 2.5% v/v DIPEA was added. After stirring for 5 minutes the solution was transferred to a peptide synthesis vessel containing resin-bound S^{t}Bu protected peptide. After 16 h the resin was filtered and washed exhaustively with DMF and then DCM. This procedure was repeated twice.

### General Bromoacetamide Couplings.

Bromoacetamides (3 eq. per thiol: 0.05 mmol×2 thiols×3 eq.=0.3 mmol) were dissolved in DMF (400 μL) and NEt₃ (65 μL, 0.45 mmol) and transferred to a peptide synthesis vessel containing resin-bound S^{t}Bu deprotected peptide. The reaction was allowed to proceed for 24 h. After this time, the resin was filtered and washed exhaustively with DMF and the DCM.

### General Alloc Deprotection.

The resin was washed for 5 min with a solution of DMF/CHCl₃/AcOH/N-methyl morpholine (NMM) (18.5:18.5:2:1). Pd(PPh₃)₄ (115 mg, 0.1 mmol) was dissolved in a solution of DMF/CHCl₃/AcOH/NMM (18.5:18.5:2:1) (0.05 M). The mixture was under nitrogen for 2 h with exclusion of light. The resin was then washed sequentially with the following solutions: 0.5% DIPEA in DMF (v/v) (4×2 ml); DMF (6×2 ml); 0.5% sodium diethyldithiocarbamate trihydrate in DMF (w/v) (4×2 ml); followed by a final wash with DMF (6×2 ml).

### Coupling of 2-acetamido-2-deoxy-3,6-diacetyl-4-carboxymethyl-β-D-glucopyranosyl azide

The resin was washed with DMF (2 ml). Coupling reactions with 2-acetamido-2-deoxy-3,6-diacetyl-4-carboxymethyl-β-D-glucopyranosyl azide was conducted using 0.25 mmol (5 equiv.) of the sugar, HBTU/HOBt and DIPEA for 3 h.

### Cleavage of Compound A.

The resin was washed with DCM and DMF five times (5 min each wash). The compound A was cleaved from the resin by incubation with the cleavage cocktail (95% TFA, 2.5% water and 2.5% EDT) for 3 h. The resin was subsequently washed twice with the cleavage cocktail (10 min each wash). The compound A was precipitated with cold diethyl ether and centrifuged for 10 min. The precipitate was redissolved in a solution 1:1 water/acetonitrile and lyophilized.

### Compound A Purification.

The crude product was purified by reverse phase preparative HPLC-MS (water-acetonitrile, 0.1% TFA).

The purified product was 19 mg (18% overall yield of 12 steps).

Analytical data of compound A: C₈₄H₁₂₁N₁₃O₄₆S₂. MW: 2113.05. Found: (M+1) 2113.05, (M+2) 1057.52

### Synthesis of Compound B:

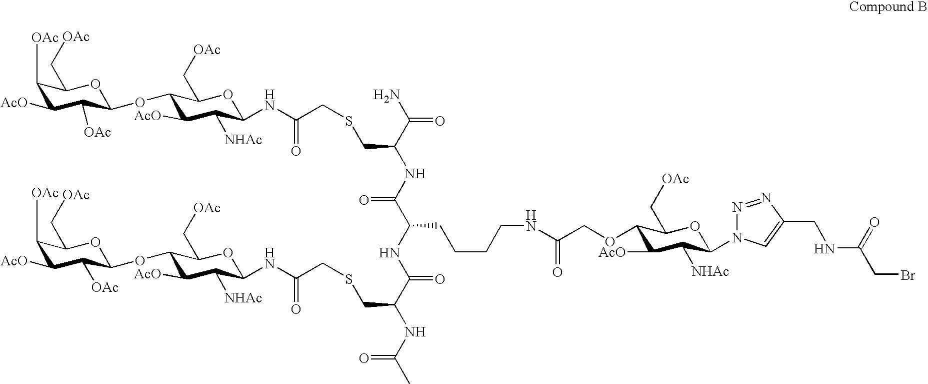

The compound A (5 mg, 0.0023 mmol) and propargyl bromoacetamide (0.5 mg, 0.0023 mmol) were dissolved in a biphasic solution of CHCl₃/EtOH/H₂O (9:1:1) (220 μL). Sodium ascorbate (0.5 mg, 0.0023 mmol) and CuSO₄.5H₂O (traces, 0.0002 mmol) were added. The reaction was stirred at 600 rpm, 37° C. overnight. Afterwards, the reaction was diluted with ethyl acetate and washed with saturated aqueous NaHCO₃ (3×10 ml), and the organic phase was dried with MgSO₄, filtered and concentrated under reduced pressure to afford the product (5 mg, 90%).

Analytical data: C₈₉H₁₂₇BrN₁₄O₄₇S₂. MW: 2289.06. Found: (M+2) 1146.15.

### Example 3

### Experimental Details for Example 3

### Instrumentation:

¹H NMR spectra were recorded at 250 and 300 MHz, ¹³C NMR spectra were recorded at 63 and 75 MHz and ¹⁹F NMR spectra were recorded at 235 MHz on a Bruker 250Y instrument. Chemical shifts (δ) were reported in ppm and coupling constants (J) in Hz, signals were sharp unless stated as broad (br), s: singlet, d: doublet, t: triplet, m: multiplet and q: quaternary. Residual protic solvent, CDCl₃ (δ_{H}: 7.26, s) was used as the internal standard in ¹H-NMR spectra unless otherwise stated. Electrospray mass spectroscopy was carried out on a Micromass Quattro LC electrospray with an applied voltage of 25-60V.

### Chromatography

Analytical TLC was carried out on Merck aluminium backed plates coated with silica gel 60F₂₅₄. Flash chromatography was carried out over Fisher silica gel 60 Å particle size 35-70 micron. Components were visualized using p-anisaldehyde dip and UV light (254 nm).

### Solvent and Reagents

All reagents and solvents were standard laboratory grade and used as supplied unless otherwise stated. Where a solvent was described as dry it was purchased as anhydrous grade. All organic extracts were dried over anhydrous magnesium sulphate prior to evaporation under reduced pressure.

### N-(propargyl)-bromacetamide

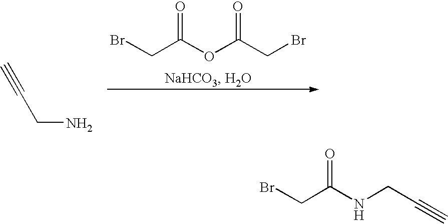

Propargylamine (0.20 mL, 2.90 mmol) was dissolved in water (30.00 mL) and immediately was treated with bromacetic anhydride (3.70 g, 14.50 mmol) in the presence of NaHCO₃ (5.00 g). The reaction was stirred at room temperature for 16 h. The reaction was quenched with 5% aqueous HCl (50.00 mL) and the product was extracted with ethylacetate (3×50.00 mL). The organic phase wash washed with NaOH 1 N (5×100.00 mL) and water (2×100.00 mL). The organic phase was dried with MgSO₄, filtered and concentrated under reduced pressure to afford a white crystal (0.38 g, 76%). R_{f}: 0.33 (petroleum ether/ethyl acetate 1:1). ¹H-NMR (250 MHz, CDCl₃) δ (ppm): 6.78 (1H, bp, NH); 4.07 (2H, q, J=5.4 Hz, J=2.6 Hz, CH₂); 3.88 (2H, s, COCH₂Br); 2.27 (1H, t, J=2.6 Hz, CH). ¹³C-NMR (63 MHz, CDCl₃) δ (ppm): 165.3 (qC, CO); 78.5 (qC, alkyne); 72.2 (CH), 29.9 and 28.6 (CH₂). FAB-MS calculated for C₅H₆BrNO (M+1) 174.96, found: 197.85 and 199.85 (M+23).

### L-Cysteine-S—(N-propargyl)-carboxymethylamide

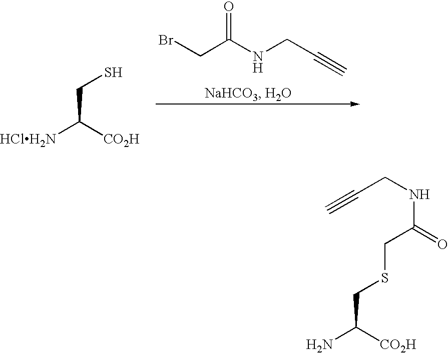

L-cysteine hydrochloride (268 mg, 1.70 mmol) was dissolved in water (2.0 ml) and 2-bromoacetyl propargylamide (300 mg, 1.70 mmol) was added. Solid sodium hydrogen carbonate was added in small portions (with evolution of CO₂) until pH 8.0 was established. The reaction mixture was then stirred at room temperature for a further 2 h. The reaction mixture was then frozen in liquid nitrogen and lyophilised to afford the crude product as a pale brown solid and was used without further purification, ¹H-NMR (300 MHz, D₂O) δ (ppm): 3.99 (2H, d, J=2.5 Hz, CH₂N); 3.93 (1H, q, αCH, J=7.7 Hz, J=4.3 Hz); 3.37 (2H, s, C(O)CH₂S), 3.17 (1H, dd, J=14.8 Hz, J=4.3 Hz, CH₂S), 3.06 (1H, dd, J=14.8 Hz, J=7.7 Hz, CH₂S), 2.61 (1H, t, J=2.5 Hz, CH).

### N-α-(9-fluorenylmethoxycarbonyl)-L-Cysteine-S—(N-propargyl)carboxymethylamide

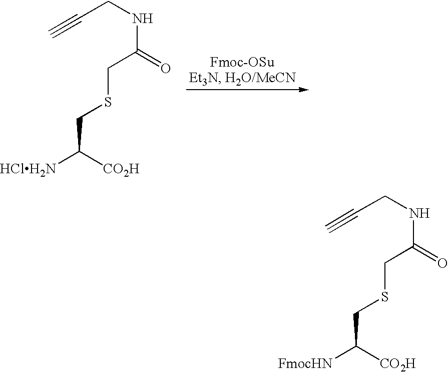

The crude propargylamide was dissolved in water (2.0 ml) and Et₃N (146 μl) was added. Fmoc-succinimide (336 mg) was dissolved in acetonitrile (2.0 ml) and this solution was added in one portion to the aqueous amino acid solution and stirring was continued at room temperature for a further 1.5 h. The pH of the reaction was monitored throughout the reaction to ensure it remained between 8 and 9, adding further Et₃N as required. After 1.5 h the reaction mixture was evaporated to dryness and the residue was partitioned between dichloromethane (30.0 ml) and 2 M HCl (30.0 ml). The organic phase was separated and the aqueous phase was extracted with dichloromethane (1×30.0 ml). The combined organic extracts were washed with 2 M HCl (25.0 ml) and sat. aq. NaCl (25.0 ml), dried with MgSO₄, filtered and the solvent was removed under vacuum to afford the crude product as an off-white solid. The crude product was purified by flash column chromatography over silica (a short column: 5 cm silica, eluent 100% EtOAc then 20% MeOH in EtOAc to afford the pure product (370 mg, 47%) as a white foam. Rf=0.05 (4:1 EtOAc/MeOH), ¹H-NMR (400 MHz, CDCl₃) δ (ppm): 7.77-7.26 (8H, m, ArH); 4.41-4.27 (3H, m, CHCH₂O and αCH); 3.96 (2H, d, J=2.5 Hz, CH₂N); 3.23 (2H, s, COCH₂S); 3.11 (1H, dd, J=13.9 Hz, J=4.5 Hz, CH₂S), 2.93 (1H, dd, J=13.3 Hz, J=8.1 Hz, CH₂S), 2.56 (1H, t, J=2.5 Hz, CH). ESI-MS calculated for C₂₃H₂₂N₂O₅S 438.1249. found: 461.18 [MNa]⁺.

### Synthesis of Compound C:

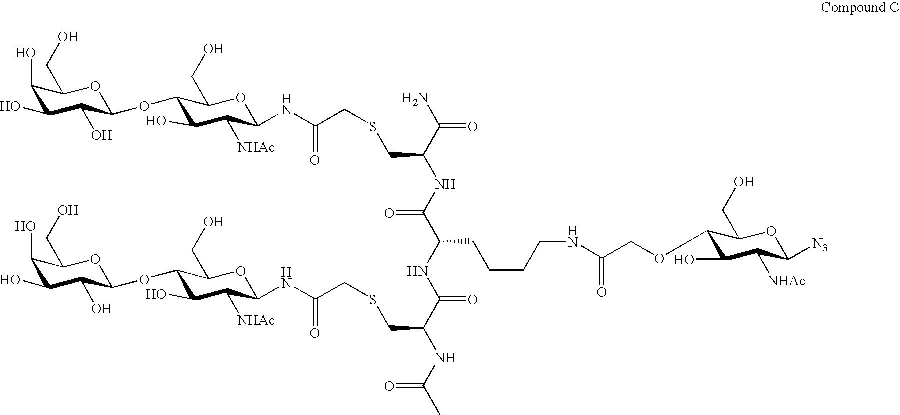

### General.

Solid-phase peptide synthesis was carried out manually using a plastic syringe fitter with a Teflon filter and connected to a vacuum waste chamber via a Teflon valve. Synthesis of the glycopeptide was carried out on MBHA Rink amide resin (loading=0.58 mmol/g).

### General Solid-Phase Peptide Synthesis.

The scale of the synthesis of compound C was 0.10 mmol. The Fmoc group was removed by treatment with 20% (v/v) piperidine in DMF (5 and 15 min) between each coupling and deprotection step. Coupling reactions with Fmoc amino acids were conducted using 0.50 mmol (5 equiv.) of the amino acid, HBTU/HOBt and DIPEA for 3 h. The reaction progress was monitored using the Kaiser ninhydrin test. Between each coupling and deprotection, the resin was washed with DCM and DMF (5 min each).

### General StBu Deprotection.

DTT (200 mg) was dissolved in dry DMF (1.8 mL) and 2.5% v/v DIPEA was added. After stirring for 5 minutes the solution was transferred to a peptide synthesis vessel containing resin-bound S^{t}Bu protected peptide. After 16 h the resin was filtered and washed exhaustively with DMF, mixtures of DMF/H₂O (1:1), DMF and finally DCM. This procedure was repeated twice.

### General Bromoacetamide Couplings.

Saccharide bromoacetamide (2 eq. per thiol: 0.10 mmol×2 thiols×2 eq.=302 mg, 0.40 mmol) was dissolved in DMF (1.0 mL) and NEt₃ (84 μL, 0.6 mmol) and transferred to a peptide synthesis vessel containing resin-bound S^{t}Bu deprotected peptide. The reaction was allowed to proceed for 24 h. After this time, the resin was filtered and washed exhaustively with DMF and the DCM.

### General Alloc Deprotection.

The resin was washed for 5 min with a solution of DMF/CHCl₃/AcOH/N-methyl morpholine (NMM) (18.5:18.5:2:1). Pd(PPh₃)₄ (230.00 mg, 0.2 mmol) was dissolved in a solution of DMF/CHCl₃/AcOH/NMM (18.5:18.5:2:1) (0.05 M). The mixture was under nitrogen for 2 h with exclusion of light. The process was repeated again. Finally, the resin was then washed sequentially with the following solutions: 0.5% DIPEA in DMF (v/v) (4×2.00 mL); DMF (6×2.00 mL); 0.5% sodium diethyldithiocarbamate trihydrate in DMF (w/v) (4×2.00 mL); followed by a final wash with DMF (6×2.00 mL).

### Coupling of 2-acetamido-2-deoxy-3,6-diacetyl-4-carboxymethyl-β-D-glucopyranosyl azide and peracetylation of the molecule

The resin was washed with DMF (2.00 mL). Coupling reactions with 2-acetamido-2-deoxy-3,6-diacetyl-4-carboxymethyl-β-D-glucopyranosyl azide (97 mg, 0.25 mmol, 2.5 equiv.), 2.5 equiv. PyBOP/HOBt and 5 equiv. DIPEA for 16 h. The process was repeated. Afterwards the resin was exhaustively washed with DCM and DMF.

Cleavage of Compound C from the Solid Support.

The resin was washed with DCM and DMF five times (5 min each wash). The fully protected precursor was cleaved from the resin by incubation with the cleavage cocktail (95% TFA, 2.5% water and 2.5% EDT) for 3 h. The resin treated again with the same cleavage cocktail for another 3 h. Compound C was precipitated with cold diethyl ether and centrifuged for 10 min. The precipitate was dried by flushing nitrogen for 30 min. The precipitate was resuspended with a water/methanol solution (10:1) of 5% hydrazine (6.0 ml). The reaction was stirred at room temperature for 24 h.

### Compound C Purification.

The crude product was purified by reverse phase semi preparative HPLC (water-acetonitrile 5%-95% over 40 minutes, 0.1% TFA) and obtained as a fluffy white solid (35.9 mg, 47% yield based on resin loading) after lyophilisation. Analytical data of compound C: C₅₆H₉₃N₁₃O₃₂S₂. MW: 1524.79. Found: (M+1) 1525.79; (M+2) 763.48; (M+2+NH₂NH₂) 779.40; 795.63 (M+2+2NH₂NH₂). The results are shown in FIG. 11.

Production of bacterially derived erythropoietin fragments was carried out as previously described:

Depletion of Residual His₁₀-Tagged EPO 28/33-166 after CNBr Cleavage

Where incomplete CNBr cleavage of HIS₁₀-tagged EPO fragments was observed, EPO preparations were resuspended in 1 ml of resuspension buffer (6 M guanidine hydrochloride containing 20 mM Tris-HCl pH 8.0, 500 mM NaCl, 5 mM imidazole, 1 mM PMSF) and mixed with 200 μl of pre-equilibrated His-Bind resin (Novagen). Samples were mixed on ice for 4 hours and resin pelleted by centrifugation. The supernatant was collected and protein precipitated out of solution by dialysis against water at 4° C. for 24 hours in 8 kDa molecular weight cut-off dialysis bags. Precipitated protein was collected by centrifugation.

### Peptide Synthesis

Peptide synthesis was carried out using Rink amide-MBHA-LL resin for the production of peptide thioesters (loading=0.34 mmol/g). All resins and Fmoc amino acids were purchased from Novabiochem. Mass spectra were obtained on a Micromass Quattro LC series electrospray mass spectrometer. Semi-preparative HPLC was performed using a Phenomenex LUNA C₁₈ column and a gradient of 5-95% acetonitrile containing 0.1% TFA over 45 minutes (flow rate of 3.0 mL/min). All other chemical reagents were obtained form Aldrich.

### Peptide Thioester Synthesis (EPO Residues 1-28).

The peptide thioesters were prepared using the dual linker strategy recently described by the Unverzagt group. Briefly, rink amide resin (0.1 mmol) was deprotected by exposure to 20% piperidine in DMF. Fmoc-Phe-OH (5 equiv) was coupled using HBTU/HOBt as coupling reagents. The coupling time was 4 h. After Fmoc removal with 20% piperidine in DMF the sulfonamide linker was coupled through exposure of the resin to 3-carboxypropanesulfonic acid (50 mg, 0.3 mmol), HOBt (40 mg, 0.3 mmol) and DIC (47 μL, 0.3 mmol) for 5 h. The first amino acid (Fmoc-Gly-OH, 5 equivalents per coupling) was then double coupled employing N-methylimidazole (40 μL, 0.5 mmol), DIC (78 μL, 0.5 mmol) as coupling reagents in 4:1 DCM/DMF for 16 h. The peptide (0.05 mmol scale) was extended (target sequence: APPRL(I*)CDSR(V*)L(E*) RYLL(E*)A(K*E*A*E*)C(I*)TTG-SBn) and cleaved with benzylmercaptan, after ICH₂CN activation, using well established procedures.² The residues with an adjacent asterisk were double coupled and the bold and underlined cysteine residues were introduced as the Fmoc-acetylenic cysteine derivatives described above. The crude fully deprotected and precipitated peptide was redissolved in 25% aqueous MeCN and purified by semi-prep HPLC. The major peak (retention time=26 mins, 6 mg, 5.3%) was analysed by ESI-MS and was found to correspond to the desired product. This fraction was lyophilized and used in subsequent NCL reactions (FIG. 12).

### Protein Assembly:

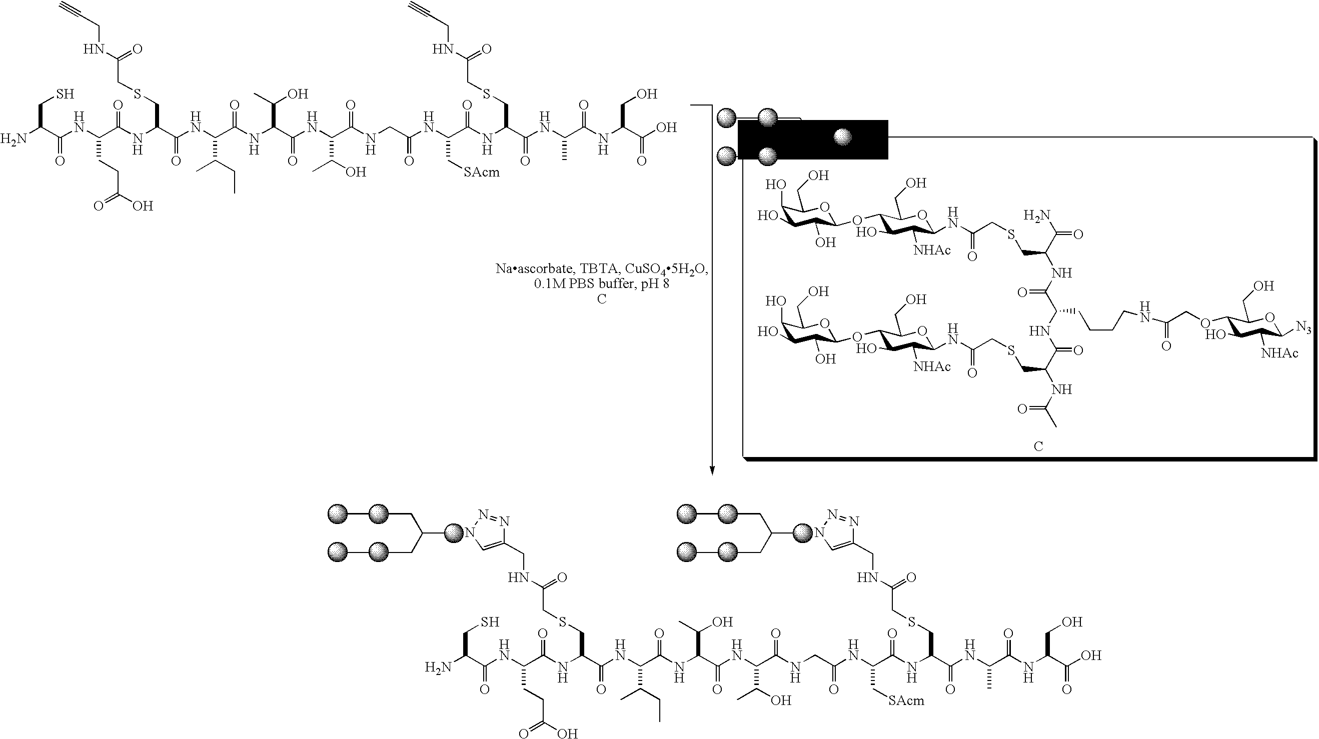

In a model reaction the acetylene-modified peptide (3.60 mg, 2.67·10⁻³ mmol) and C (14.30 mg, 9.38·10⁻³ mmol) were dissolved in a 0.1M PBS buffer solution pH 8 (1.90 mL). Sodium ascorbate (13.00 mg, 6.66·10⁻² mmol) and tris-benzyltriazolylmethylamine (TBTA) (35.00 mg, 6.66·10⁻² mmol) were added to the solution. The final suspension was sonicated for 5 minutes in a cold bath of water. Finally, a PBS solution of CuSO₄.5H₂O (0.67 mg, 2.67·10⁻³ mmol, 26.70 mM, 100 μL) was added. The reaction was shaken for 24 hours at 20° C. and 1200 rpm in an Eppendorf thermomixer. Then, the crude reaction centrifuged at 14000 rpm for 5 minutes, and filtered through a celite plug (in a Pasteur pipette). The crude product was purified by reverse phase semi preparative HPLC (water-acetonitrile 5%-95% over 40 minutes, 0.1% TFA). The corresponding fractions were freeze dried to afford a fluffy white solid (t_{R}=16.1 min) (8.10 mg, 69%), and a white fluffy white solid C (t_{R}=14.2 min) (5.10 mg recovered starting material).

Analytical data: C₁₆₄H₂₆₈N₄₀O₈₄S₈. MW: 4400.6. Found: (M+3) 1468.6, (M+4 (+K)) 1111.5, (M+4) 1101.7, (M+5) 881.7, (M+2 (saccharide mimic-N₃)) 741.9 (FIG. 13).

### Protein Thiol Modification Employing a Triazole-Linked Bromoacetamide:

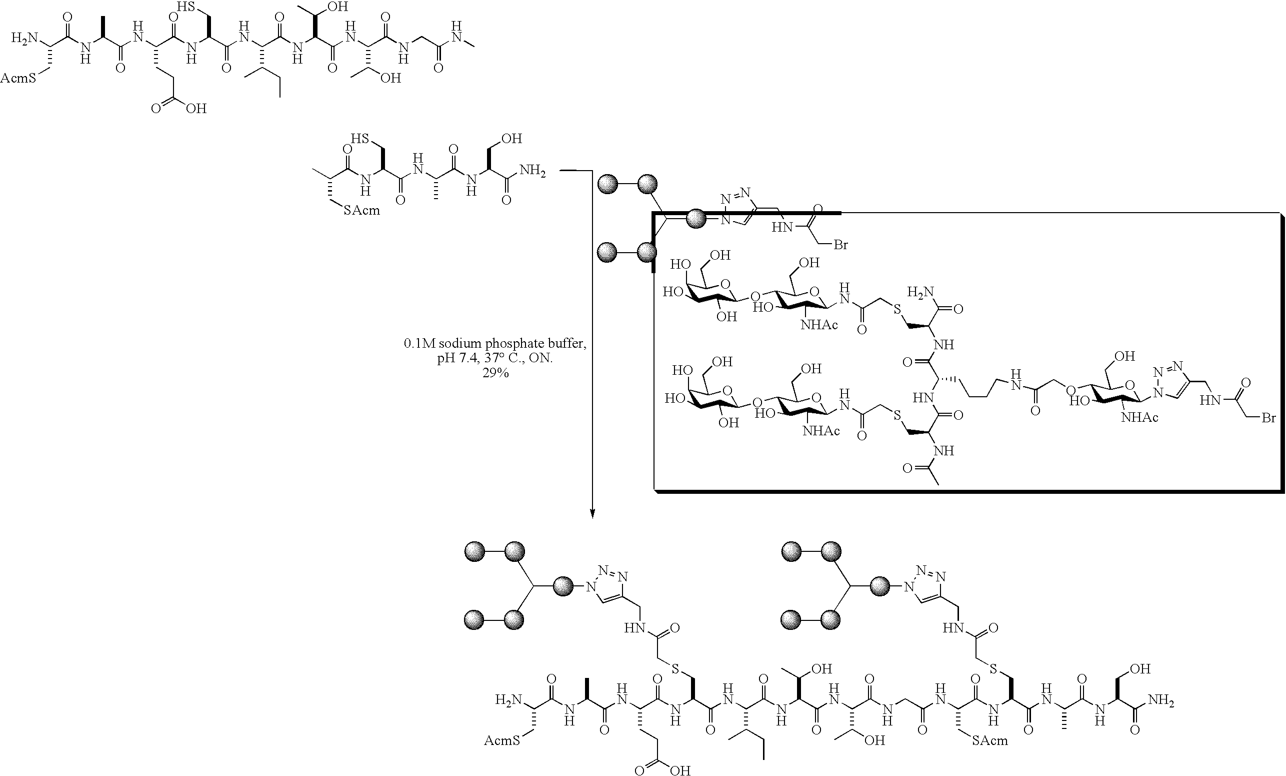

The desired dithiol containing peptide (1.50 mg, 1.15·10⁻³ mmol) and the bromoacetamide (9.40 mg, 5.53·10⁻³ mmol) were dissolved in a 0.1M sodium phosphate buffer solution, pH 7.4 (400.00 μL). The mixture was incubated at 600 rpm, 37° C., overnight. The crude product was purified by reverse phase semi preparative HPLC (water-acetonitrile 5%-95% in 40 minutes, 0.1% TFA). The corresponding fractions were freeze-dried to afford a fluffy white solid (1.70 mg, 29%). Analytical data: C₁₇₀H₂₇₉N₄₃O₈₅S₈. MW: 4541.80. Found: (M+3 (+K)) 1528.0, (M+4 (+2K)) 1156.0, (M+5 (+2K)) 925.0, (M+6 (+3K)) 777.5

### SDS PAGE and Western Blotting

Preparations of erythropoietin were precipitated by addition of 20 sample volumes of methanol and acetone solution (1:1 v/v), incubated at −20° C. for 30 minutes and spun out of solution by centrifugation. Samples were resolubilised in 20 μl of 8 M urea and boiled with loading buffer. Proteins were resolved by SDS PAGE on pre-cast 18% polyacrylamide gels as described. Electrophoresed proteins were visualised by Coomassie staining. For Western blotting, electrophoresed proteins were transferred to nitrocellulose membrane using the TransBlot transfer system (BioRad). Membranes were blocked in blocking solution (16 mM Na₂HPO₄, 4 mM NaH₂PO₄, 100 mM NaCl, 0.1% (v/v) tween-20, 5% (w/v) fat free milk powder) and probed with anti-human erythropoietin monoclonal antibody (R & D systems) diluted in the same solution. Unbound antibodies were washed off with blocking solution (without milk powder). Membranes were incubated with peroxidase-conjugated anti-mouse IgG (Sigma), washed as described above and proteins visualised using the SigmaFast 3,3′-diaminobenzidine tetrahydrochloride system (Sigma).

### Native Chemical Ligation Reactions:

Human erythropoietin (for example, residues 29-166 or 33-166) were expressed, purified and prepared for ligation reactions as described above. 3 mg of protein was dissolved in degassed ligation buffer (300 mM NaHPO₄; pH 8.0, 6 M guanidine hydrochloride, 1% w/v MESNa, 40 mM TCEP, 333 μl). This solution was combined with 3 mg of synthetic peptide thioester in ligation buffer (500 μl) and the reaction was agitated at 37° C. Samples (10 μl) were analysed by SDS-PAGE at 0, 48, 96, and 144 hours. The ligation was readily observed employing a commercial, available anti-hEPO monoclonal antibody which recognises a continuous epitope within the first twenty five amino acid residues. The western blot below confirms that the ligation proceeds (note that at t=0 the western blot is negative as the bacterial fragment (29-166) does not contain the required epitope). After as little as 1 h, product formation at the correct molecular weight of 18 kDa is observed (see FIG. 14).

1. A glycopeptide of the formula S-L-X—P, wherein:
S is selected from an optionally protected monosaccharide, an optionally protected polysaccharide, a polyalkylene oxide chain and a group of the formula II 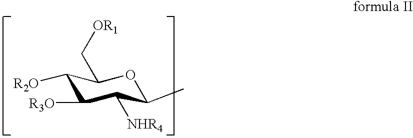
wherein R₁ and R₃ are independently selected from H or Ac,
R₄ is Ac,
and R₂ is a group of formula IV 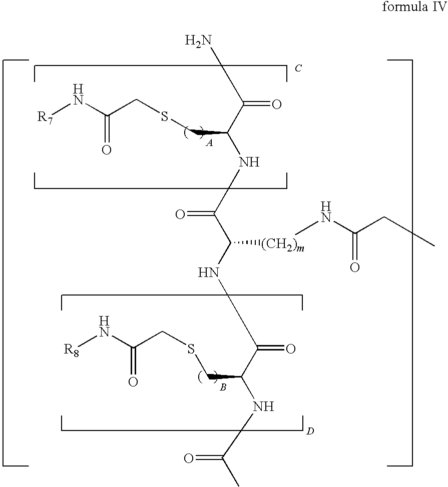
wherein R₇ and R₈ are each independently selected from an optionally protected monosaccharide and an optionally protected polysaccharide,
A, B, C and D are each independently 1 or 2, and
m is 1 to 5;
-L- is a moiety of the formula III 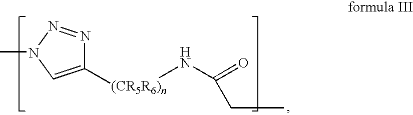
wherein R₅ and R₆ are independently selected from H and Me and n is 1 to 3; and
P is a peptide chain containing at least one amino acid having on its side chain the atom X, wherein X is an oxygen, a sulphur atom or a —CH₂— moiety.

2. A glycopeptide as claimed in claim 1, wherein S— is a moiety of the formula II 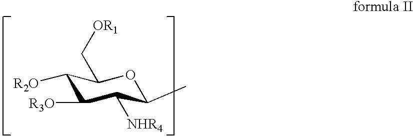
wherein R₁ and R₃ are independently selected from H or Ac,
R₄ is Ac, and R₂ is a group selected from H, an optionally protected monosaccharide, an optionally protected polysaccharide and Ac.

3. A glycopeptide as claimed in claim 2, wherein R₂ is H or Ac.

4. A glycopeptide as claimed in claim 2, wherein R₂ is an optionally protected monosaccharide is selected from glucose, glucosamine, galactose, N-acetylglucosamine, galactosamine, mannose, fucose and sialic acid.

5. A glycopeptide as claimed in claim 2, wherein R₂ is an optionally protected polysaccharide containing from 2 to 5 component sugars and comprising one or more of glucose, glucosamine, galactose, N-acetylglucosamine, galactosamine, mannose, fucose and sialic acid.

6. A glycopeptide as claimed in claim 1, wherein R₂— is a moiety of formula IVA 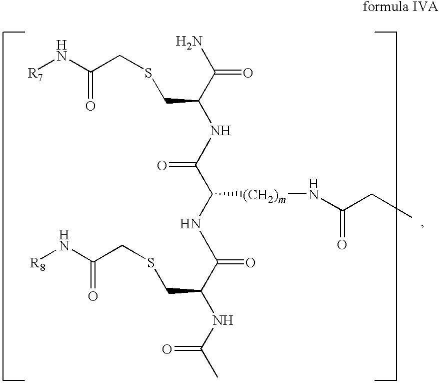
wherein R₇ and R₈ are each independently selected from an optionally protected monosaccharide and an optionally protected polysaccharide and m is 1 to 5.

7. A glycopeptide as claimed in claim 1, wherein R₇ and/or R₈ is an optionally protected polysaccharide containing from 2 to 5 component sugars and comprising one or more of glucose, glucosamine, galactose, N-acetylglucosamine, galactosamine, mannose, fucose and sialic acid.

8. A glycopeptide as claimed in claim 1, wherein R₇ and/or R₈ is an optionally protected disaccharide comprising one or more of glucose, glucosamine, galactose, galactosamine, mannose, fucose and sialic acid.

9. A glycopeptide as claimed in claim 7, wherein R₇ and/or R₈ is a disaccharide comprising N-acetylglucosamine and galactose, wherein galactose is the terminal sugar component of the disaccharide.

10. A glycopeptide as claimed in claim 1, wherein the at least one amino acid is cysteine.

11. A method of synthesising a glycopeptide as defined in claim 1, the method comprising:
contacting a peptide of formula H—X—P with a compound of formula S-L-Hal, in the presence of a base to form S-L-X—P, wherein S, L, and P are as defined in claim 1, X is an oxygen or a sulphur atom, and Hal is Br or I.

12. A method of synthesising a glycopeptide as defined in claim 1, the method comprising:
contacting a compound of the formula S—N₃ with a compound of the formula HC≡C-L^{p}-X—P in the presence of a suitable catalyst to form S-L-X—P, wherein S, L, X and P are as defined in claim 1, -L^{P}- is a moiety of the formula VII 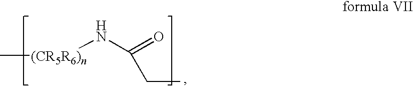
and R₅, R₆ and n are as defined in claim 1.

13. A compound for linkage to a peptide, the compound having the formula I
S-L-Hal formula I
wherein S— is a moiety of the formula II or a polyalkylene oxide chain 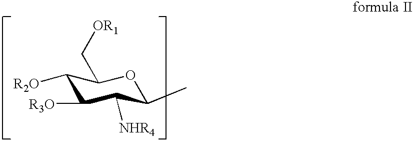
-L- is a moiety of the formula III 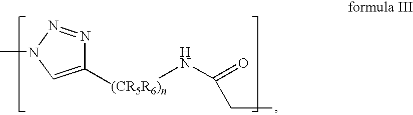
and Hal is Br or I;
wherein R₁ and R₃ are independently selected from H and Ac,
R₄ is Ac,
R₅ and R₆ are independently selected from H and Me,
n is 1 to 3, and
R₂ is a group selected from H, an optionally protected monosaccharide, an optionally protected polysaccharide, Ac, and a group of the formula IV, 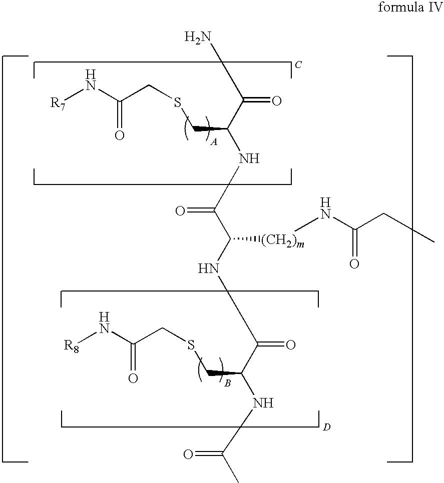
wherein R₇ and R₈ are each independently selected from an optionally protected monosaccharide and an optionally protected polysaccharide,
A, B, C and D are each independently 1 or 2, and
m is 1 to 5.

14. A compound as claimed in claim 13, wherein R₂ is H or Ac.

15. A compound as claimed in claim 13, wherein R₂ is an optionally protected monosaccharide selected from glucose, glucosamine, galactose, N-acetylglucosamine, galactosamine, mannose, fucose and sialic acid.

16. A compound as claimed in claim 13, wherein R₂ is an optionally protected polysaccharide containing from 2 to 5 component sugars and comprising one or more of glucose, glucosamine, galactose, N-acetylglucosamine, galactosamine, mannose, fucose and sialic acid.

17. A compound as claimed in claim 13, wherein R₂ is a moiety of formula IVA 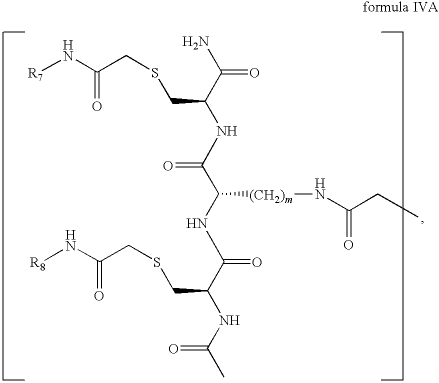
wherein R₇ and R₈ are each independently selected from an optionally protected monosaccharide and an optionally protected polysaccharide and m is 1 to 5.

18. A compound as claimed in claim 13, wherein R₇ and/or R₈ is an optionally protected polysaccharide containing from 2 to 5 component sugars and comprising one or more of glucose, glucosamine, galactose, N-acetylglucosamine, galactosamine, mannose, fucose and sialic acid.

19. A compound as claimed in claim 13, wherein R₇ and/or R₈ is an optionally protected disaccharide comprising one or more of glucose, glucosamine, galactose, galactosamine, mannose, fucose and sialic acid.

20. A compound as claimed in claim 19, wherein R₇ and/or R₈ is a disaccharide comprising N-acetylglucosamine and galactose, wherein galactose is the terminal sugar component of the disaccharide.

21. A method of synthesising a compound as defined in claim 13, the method comprising:
contacting, in the presence of a suitable catalyst, a compound of the formula V, wherein S is as defined in claim 13,
S—N₃ formula V
with a compound of the formula VI
HC≡C-L^{p}-Hal formula VI,
wherein -L^{p}- is a moiety of the formula VII 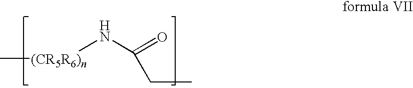
wherein R₅, R₆, n, and Hal are as defined in claim 1.

22. A method as claimed in claim 21, wherein the catalyst comprises Cu(I) or Cu(II).

23. (canceled)

24. (canceled)

25. (canceled)

26. A method of synthesising a compound of the formula HC≡C-L^{p}-X—P, wherein -L^{P}- is a moiety of the formula VII 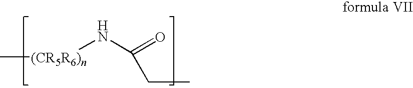
wherein R₅ and R₆ are independently selected from H and Me and n is 1 to 3,
wherein P is a peptide chain containing at least one amino acid having on its side chain the atom X, wherein X is an oxygen or sulphur atom,
the method comprising:
contacting an amino acid having at least one atom X on its side chain, with a compound of formula HC≡C-L^{p}-Hal, wherein Hal is Br or I, to form a HC≡C-L^{p}-X— functionalised amino acid,
and using said functionalised amino acid in peptide chain assembly to form HC≡C-L^{p}-X—P.

27. A method of synthesising a compound of the formula HC≡C-L^{p}-X—P, wherein -L^{P}- is a moiety of the formula VII 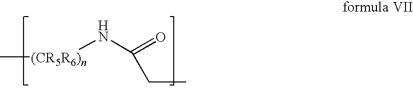
wherein R₅ and R₆ are independently selected from H and Me and n is 1 to 3;
the method comprising:
providing a peptide chain P, wherein P is a peptide chain containing at least one amino acid having on its side chain the atom X, wherein X is an oxygen or sulphur atom,
and contacting the peptide chain with a compound of formula HC≡C-L^{p}-Hal, wherein Hal is Br or I, to form
HC≡C-L^{p}-X—P.


SEQUENCE LISTING (2 sequences)

SEQ ID NO: 1 - protein, 167 aa, Artificial Sequence
  Modified Erythropoietin
CAPPRLICDSRVLERYLLEAKEAECITTGCCESCSLNENITVPDTKVNFYAWKRLEVGQQAVEVWQGLALLSEAVLRGQALLVKSSQPWEPLQLHVDKAVSGLRSLTTLLRALGAQKEAISPPDAAKAAPLRTITADTFRKLFRVYSNFLRGKLKLYTGEACRTGDR

SEQ ID NO: 2 - protein, 32 aa, Artificial Sequence
  Synthetic Peptide
APPRLICDSRVLERYLLEAKEAECITTGCCES